(19) United States
(12) Patent Application Publication
(10) Pub. No.: US 2018/0057881 A1
(43) Pub. Date: Mar. 1, 2018

(54) METHODS OF DETECTING SNPS RELATED TO MRSA AND SSI

(71) Applicant: William Beaumont Hospital, Royal Oak, MI (US)

(72) Inventor: Matthew Sims, Troy, MI (US)

(21) Appl. No.: 15/685,449

(22) Filed: Aug. 24, 2017

Related U.S. Application Data
(60) Provisional application No. 62/378,851, filed on Aug. 24, 2016.

(51) Int. Cl. C12Q 1/68

(52) U.S. Cl. CPC C12Q 1/6883; C12Q 2600/118; C12Q 2600/156

(57) ABSTRACT

A method for identifying, diagnosing, and treating individuals who may be at risk developing or may have developed recurrent antibiotic resistant skin infections including MRSA and SSI. Also included are methods for detecting whether a nucleotide of one or more single nucleotide polymorphisms (SNP) is present at a SNP position, related probes.

### FIELD OF INVENTION

The present invention is in the field of biochemistry and medicine and relates to methods for detecting, diagnosing, and/or treating Antibiotic-Resistant Staphylococcus aureus (MRSA) and Surgical Site Infection (SSI).

### CROSS-REFERENCE TO RELATED APPLICATION

This U.S. patent application claims priority to U.S. Provisional Application 62/378,851 filed on Aug. 24, 2016, the disclosure of which is considered part of the disclosure of this application and is hereby incorporated by reference in its entirety.

### SEQUENCE LISTING

The Sequence Listing accompanying this application is contained within the computer readable file entitled “2016-06-14_5475-373745_SEQ_LIST_ST25.txt” submitted electronically and contemporaneously with the filing of this application through the USPTO EFS-Web. The file is 2.03 MB, was created on Jun. 14, 2016, and is incorporated herein by reference.

### BACKGROUND OF THE INVENTION

Staphylococcus aureus (Staph, S, or SA) bacteria are a common component of the skin surface and lining of the nasal passageways in humans and other animals. Staph aureus is usually spread by skin-to-skin or skin-to object-to skin contact. Studies show that about one in three people carry Staph in their nose according to the U.S. Centers for Disease Control (CDC). When limited to the skin surface and lining of the nasal passageways, S. aureus bacteria are normally harmless. However, S. aureus infections can occur in situations where the bacteria enter into the skin subsurface or body cavity, normally through wounds (including, e.g., surgical incisions) or other sites such as hair follicles. Methicillin-Resistant Staphylococcus aureus is known as, and is used here by the abbreviation “MRSA.” MRSA is a strain of Staph. aureus that has become resistant to methicillin and other related antibiotics, commonly used to treat ordinary S. aureus infections. Studies show that about 2 in 100 people carry MRSA, according to the CDC. MRSA infections are particularly hard to treat and a subset of the population we call “MRSA high risk” is at higher than normal risk of getting a MRSA infection following exposure to the MRSA bacteria.

SSI is another type of infection that is often caused by MRSA. SSI is an abbreviation for “Surgical Site Infection.” SSI can appear as an abscess or inflammation of the skin often associated with a wound, usually a surgical wound. An SSI is often the direct or indirect result of surgery and frequently but not always involves a MRSA infection.

MRSA infections and SSIs can start as swollen, painful red bumps that might resemble pimples or spider bites. They can appear as swollen and painful boil-like symptoms, and they are typically initially treated locally. The affected area can be warm to the touch, full of pus or other drainage and accompanied by a fever. The “red bumps” can quickly turn into deep, painful abscesses. Sometimes the bacteria remain confined to the skin. But they can also burrow deep into the body, causing life-threatening infections in bones, joints, surgical wounds, the bloodstream, heart valves and lungs. MRSA sometimes causes a person's death. The CDC tracks and reports MRSA infections and deaths on its website, look up MRSA Home. MRSA and SSI Staph infections may be systemic and should usually be treated with systemically administered antibiotics; many individuals need to be treated by what is often called “decolonization” following an infection.

Traditional risk assessment for MRSA includes various forms of MRSA including the forms known as “HA” or “HS” (Hospital Acquired or Healthcare Settings). MRSA infections often involve several factors. Whether or not hospitalization is involved is important but not the only factor to be considered. MRSA infections in people exposed to general group exposure such as contact sports may have what is termed “Community-Acquired” MRSA or “CA-MRSA.” The detailed teaching of this disclosure applies to all forms of MRSA.

MRSA is a big concern in hospitals where it can attack those most vulnerable who tend to be older adults and people with weakened immune systems. Another factor is whether or not an invasive medical device is involved with or without invasive surgery. MRSA is often acquired or develops in medical care facilities, such as hospitals. This type of MRSA is known as Health Care-Associated MRSA (HA-MRSA). Devices like medical tubing, such as intravenous lines or urinary catheters, can provide a pathway for MRSA to travel into a body. MRSA is prevalent in nursing homes and thus living in a long-term care facility can increase the risk of exposure and infection. MRSA infections often occur in patients having weakened immune systems who are exposed to the bacteria, such as patients in long term care, patients undergoing kidney dialysis, and patients recovering from recent surgery or medical treatments such as chemotherapy that weaken the immune system. MRSA can spread easily through cuts and abrasions and skin-to-skin contact, thus a risk factor for both HA-MRSA and CA-MRSA.

MRSA can also develop in otherwise healthy people not exposed to hospital situations. CA-MRSA is often acquired from participation in contact sports, sharing towels, washcloths, razors, uniforms or clothing or living in crowded or unsanitary conditions and being exposed to others wounds, bandages or coverings. Skin wounding events or other forms of interruption in skin integrity (e.g., intravenous drug use) are another major risk factor for MRSA infections. Sometimes compromised individuals who are frequently exposed to MRSA never acquire the infection. Some people who do become infected with MRSA never show symptoms, but these “carriers” of MRSA can spread it without being ill themselves. CA-MRSA has often been found to be acquired by athletes (who may share towels and razors), children in day care, members of the military and people obtaining tattoos, for example. Outbreaks of MRSA have occurred in military training camps, child care centers and jails.

Until recently, the risk of developing MRSA was predicted based on a person's behavior, history, living circumstance, lifestyle and potential exposure to the resistant bacteria. It has long been known that some individuals seem to be very susceptible to Staph infections and they develop recurrent MRSA skin infections, or SSI, even though they don't have many or even any of the known risk factors. Until now, there have been no known methods to identify such people. Such populations can have an elevated risk for developing MRSA if admitted to medical care facilities in spite of not having any known MRSA risk factors. Conversely many individuals with known MRSA risk factors never develop MRSA or SSI in spite of high exposure including many lengthy stays in hospitals with invasive surgery. In this document the teaching and claims applies to and can be used with and for diseases and individuals having, exposed to or susceptible to SSI, MRSA, CA-MRSA and HA-MRSA unless indicated otherwise.

Once identified as being at high risk or as having MRSA or SSI then the treatment for these individuals can involve attempts to decolonize the skin and nasal passages of the patient with topical antibiotics. This can be temporarily effective, especially when coupled with improved sanitation such as frequent hand washing and isolation from other patients, but often it fails.

Improved methods of recognizing and predicting who may be more susceptible than the average, or normal person, to recurrent MRSA, CA-MRSA, HA-MRSA and SSI are needed. We believe a person's risk of acquiring a MRSA infection can be better determined by including consideration of a person's genetic predisposition to the disease and creating a genetic and exposure based risk profile. Here we disclose new tools and new methods to make a MRSA risk analysis and MRSA treatments that are not simply based on general patterns of exposure to the disease. We provide a genetic tool based analysis with many heretofore unknown factors that we believe can both improve patient care for those susceptible to MRSA, identify those at risk of MRSA that do not have the typical risk factors, identify those NOT at risk of MRSA infections that DO have the typical risk factors. These measures can reduce the number of false positives, i.e., labeling people as being at high risk of acquiring a MRSA infection when in fact they are not at high risk. It is in no one's interest in using professional medical staff time and money to prevent a MRSA infection simply based on traditional factors, when in fact, a person is no more likely to be afflicted with a MRSA infection than the average person. We teach how to direct the efforts to prevent MRSA infections to where they need to be directed and used, on people who are truly at high risk of developing a MRSA infection.

### SUMMARY OF THE INVENTION

In one embodiment, the present invention is a method of detecting whether a nucleotide of one or more single nucleotide polymorphisms (SNP) is present at a SNP position, wherein the one or more SNPs is adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13, including: obtaining from a human subject a blood sample having or suspected of having the nucleotide at the SNP position; and detecting in the blood sample whether the nucleotide of the SNP is present at the SNP position, wherein the nucleotide is cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and/or guanine at SNP position m13. In another embodiment, the method may include detecting in the blood sample whether adenine is present at SNP position a1. And in another embodiment, the nucleotide may be detected in two or more SNPs.

In one example of the method, the subject potentially may have been exposed to or may be exposed to infection by antibiotic-resistant Staphylococcus aureus (MRSA) or a surgical site infection (SSI). In a further example: the subject may have had a surgical procedure or a procedure involving an invasive medical device, may have been living in unsanitary conditions, or may have been participating in sports activities with exposure to cuts or other injuries; or (b) may be in need of a surgery or a stay at a medical treatment or care facility.

An aspect of the invention is the method wherein the detecting step may include contacting the blood sample with an oligonucleotide probe that is hybridizable with the nucleotide located at the SNP position, and detecting the hybridization.

A further embodiment of the above-described methods also may include: administering to the subject an aggressive treatment for recurrent MRSA when the detected nucleotide of the SNP is present at the SNP position. And in an additional embodiment, the aggressive treatment may be include decolonization of MRSA from the skin or nasal passages of the subject, aggressive antibiotic therapy, administration of antibiotic-resistant drugs, isolation therapy, and/or recolonization therapy.

In another example, the present inventive method is detecting whether a nucleotide of one or more single nucleotide polymorphisms (SNP) is present at a SNP position, wherein the one or more SNPs are adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13, including: obtaining from a human patient a blood sample having or suspected of having the nucleotide at the SNP position; and detecting in the blood sample whether the nucleotide of the SNP is present at the SNP position, wherein the nucleotide is guanine at SNP position b2, thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and/or adenine at SNP position m13. And in another embodiment, the method also may include detecting in the blood sample whether cytosine is present at SNP position a1. With this embodiment, the method also may include not administering to the subject an aggressive treatment for recurrent MRSA when the detected nucleotide of the SNP is present at the SNP position.

Also disclosed herein is a method of providing medical services to a human subject suspected of having or having recurrent antibiotic-resistant Staphylococcus aureus (MRSA) or a surgical site infection (SSI), including: obtaining a biological sample from the subject; requesting diagnostic information about the sample, wherein the diagnostic information is an identity of a nucleotide of one or more single nucleotide polymorphisms (SNP) at a SNP position, wherein the one or more SNPs is adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and/or guanine or adenine at SNP position m13; and administering a treatment for recurrent MRSA or SSI when the identity of the nucleotide of one or more SNPs is a high MRSA risk SNP.

In one embodiment, the method of providing medical services further may include requesting additional diagnostic information about the sample, wherein the additional diagnostic information is an identity of adenine or cytosine at a SNP position a1; and administering a treatment for recurrent MRSA or SSI when the identity of the nucleotide of one or more SNPs is a high MRSA risk SNP and adenine is identified at the SNP position a1. In a further embodiment, the identity of the nucleotide of one or more SNPs may be cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and/or guanine at SNP position m13; and the treatment for recurrent MRSA or SSI is administered. In another embodiment, the identity of the nucleotide at SNP position a1 may be adenine; and the treatment for recurrent MRSA or SSI is administered. In another aspect, the treatment may be decolonization of MRSA from the skin or nasal passages of the subject, aggressive antibiotic therapy, administration of antibiotic-resistant drugs, isolation therapy, and/or recolonization therapy.

In another example of the method of providing medical services, the identity of the nucleotide of one or more SNPs may be guanine at SNP position b2, thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and/or adenine at SNP position m13; and the treatment for recurrent MRSA or SSI is not administered. Further, the identity of the nucleotide at SNP position a1 may be cytosine; and the treatment for recurrent MRSA or SSI is not administered.

Also described herein is an artificial probe including: one or more oligonucleotides from 5 to 1500 nucleotides in length, and conjugated to a label, marker or detectable moiety, wherein the one or more oligonucleotides is able to hybridize with from 5 to 1500 nucleotides adjacent to and including a single nucleotide located at a SNP position, and wherein the single nucleotide is cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13. The probe also may include an oligonucleotide from 5 to 1500 nucleotides in length, and conjugated to a label, marker or detectable moiety, wherein this oligonucleotide is able to hybridize with from 5 to 1500 nucleotides adjacent to and including an adenine or cytosine located at a SNP position a1.

In another aspect of the inventive probe, the one or more oligonucleotides may be from 10 to 200 nucleotides. In another example, the single nucleotide located at the SNP position may be cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and/or guanine at SNP position m13. Further, the single nucleotide located at the SNP position may be guanine at SNP position b2, thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and/or adenine at SNP position m13. As further described below, the one or more oligonucleotides may be conjugated to a detectable moiety and the detectable moiety may be a radioactive label, a P32 label; or a fluorescent label. In yet a further example of the inventive probe, the hybridization may occur under strict conditions.

In contrast to a normal or low risk individual, a “high risk” also called a “greater than normal risk” individual is someone who, once they are exposed to MRSA, will likely become infected with MRSA, or someone who has a MRSA infection, will, much more frequently than a normal person, will likely become reinfected with MRSA after standard treatment. It should be understood as we state in the background that a not insignificant portion of the population, about 1-3 in 100 individuals who, according to the CDC estimates are people who carry MRSA, often in their nostrils, but who show no symptoms and do not become infected, are so called MRSA “carriers.” In contract to those who carry MRSA but don't become infected or if infected are usually easily treated, are those more rare but not uncommon individuals, perhaps 1-3 percent of those who carry MRSA who we say are “high risk” individuals and they are either or both a) more likely to become infected in the first place from a MRSA exposure than would a normal or control person when they are simply exposed to the bacteria with typical skin contact, and b) those who once they become infected with MRSA are much more likely to become re-infected with a second or subsequent MRSA infection following a second exposure to the bacteria.

As noted above, the MRSA bacteria is not rare, and it can be found in many environments where a great many people frequent, but relatively few people (few compared to the general population) will consistently develop a MRSA infection within a month or two after being exposed to the MRSA bacteria, many of these people are also people who, once they develop a MRSA infection, are much more likely to develop a second, third or additional MRSA infection after their first infection. That is some people seem to have a much greater chance of developing repeated MRSA infections. In a relatively small group of people once they have a MRSA infection, they are likely to get another MRSA infection, often within a few months. These “high risk” or “greater than normal risk” individuals who get a MRSA infection will often go on to develop repeated MRSA infections at a much greater frequency than what is “normal” for most individuals. It is these people who are more likely to develop, or potentially may develop, initial and or repeated MRSA infections following exposure that we call “high risk” individuals.

For the first time we believe we can test, identify, predict and determine who is at high risk and treat those high risk people and we can even do it before they have had their first MRSA infection.

Furthermore some of the people who would be considered “high risk” based on traditional criteria are in fact not high risk, they do not need to endure the sometimes expensive and time consuming treatments that many high risk people are asked to accept, and we think we can identify those people as well.

Sometimes a person will have a nasty MRSA infection more than once, sometimes 3 or 4, even 8 or more times before either the person or the people treating the person will realize that the person is in fact at high risk of developing MRSA and he or she should be given extraordinary treatment. Often this extraordinary treatment will be some version of what is called “decolonization” which is a treatment with the goal of trying to eliminate any and all residual MRSA bacteria living on the person's skin. Knowing that a person is at high risk for MRSA early can save a person money, resources, time and in some cases the person's life.

We describe methods and tools that can be used to measure, predict or assess the level of risk, and even treat a subject who is developing, is likely to develop, or potentially may develop, a MRSA infection and/or an SSI infection. We describe a family of thirteen SNPs or genetic markers, we call their location in the gene the “critical SNP nucleotides” at “critical SNP positions” that, if present in an individual's genome, either individually or in combination, indicates a greater than normal risk or greater than normal chance that the individual with the high risk SNP or marker might develop MRSA or SSI if exposed to the bacteria that cause MRSA or SSI. Most of the SNPs we describe are within genes but one SNP is between two genes. The SNPs or markers we describe are in or near the following genes. FAM 129B (1 SNP); RBM6 (1 SNP); KANK 4 (1 SNP); Col 13A1—closest to gene (1 SNP); COL 19A1 (5 SNPs); IGSF8 (1 SNP); ADARB2 (1 SNP); DCT (1 SNP) and one critical SNP position is between genes LTF and CCRL-2 (1 SNP). (The number in parenthesis is the number of SNPs associated with or near the indicated gene.)

The SNPs in this document are given a unique alpha numeric number (e.g., 1a, 2b, 3c, etc.) and a unique “rs” number and those SNPs have both a “critical SNP position” and critical SNP nucleotides are in those positions. Here “critical SNP position” means the position of the nucleotide in the genome and the nucleotide at that position is then indicated herein as one of two types, it is either “normal” or “high” risk. The detection and characterization is important in determining whether or not the person being evaluated is a person who is “normal” or at “high risk” as we discuss herein and in the definitions. The critical SNP positions and nucleotides are given the following names and numbers herein and the genes, chromosomes and cytobands they are associated with. In 11 of the 13 SNPs the critical SNP position is within a gene, in 2 of the SNPs (d4 and m13) the critical SNP position is between 2 genes.

“a1)” and/or SNP/rs2249861 is a nucleotide location within the FAM 129B gene, chromosome 9, cytoband q34.11, SEQ ID NOs 1 and 2 have the full sequence of the FAM 129B gene;
“b2)” and/or SNP/rs2352969 is a nucleotide location within the RBM6 gene; chromosome 3, cytoband p21.31, SEQ ID NO 5 is the full sequence of the RBM6 gene;
“c3)” and/or SNP/rs12758910 is a nucleotide location within the KANK 4 gene; chromosome 1, cytoband p31.3, SEQ ID NO 8 is the full sequence of the KANK 4 gene;
“d4)” and/or SNP/rs2642572 is a nucleotide location between two genes, the uncharacterized protein C10orf35 isoform X1 gene and the Col 13A1 gene; chromosome 10, cytoband q22.1, SEQ ID NO 11 is the full sequence of the C10orf35 isoform X1 gene;
“e5)” and/or SNP/rs3793038 is a nucleotide location within the Col 19A1 gene; chromosome 6, cytoband q13, SEQ ID NO 14 is the full sequence of the Col19A1 gene;
“f6)” and/or SNP/rs3805987 is a nucleotide location within the Col 19A1 gene; chromosome 6, cytoband q13;
“g7)” and/or SNP/rs9454944 is a nucleotide location within the Col 19A1 gene; chromosome 6, cytoband q13;
“h8)” and/or SNP/rs978291 is a nucleotide location within the Col 19A1 gene; chromosome 6, cytoband q13;
“i9)” and/or SNP/rs978290 is a nucleotide location within the Col 19A1 gene; chromosome 6, cytoband q13;
“j10)” and/or SNP/rs1131891 is a nucleotide location within the IGSF8 gene; chromosome 1, cytoband p23.2, SEQ ID NO 25 is the full sequence of the IGSF8 gene;
“k11)” and/or SNP/rs127711411 is a nucleotide location within the ADARB2 gene; chromosome 10, cytoband p15.3, SEQ ID NO 28 is the full sequence of the ADARB2 gene;
“l12)” and/or SNP/rs4773794 is a nucleotide location within the DCT gene; chromosome 13, cytoband q32.1, SEQ ID NO 31 is the full sequence of the DCTgene; and
“m13)” and/or SNP/rs4619820 is a nucleotide location between two genes, CCR2 and LTF; chromosome 3, cytoband p21.31, SEQ ID NO 34 is the full sequence of the CCR2 gene.

Any one or a combination of two or more of the above SNPs, that is the nucleotides at the “critical SNP position” or the location in the genome of those critical SNP nucleotides, which we call the “critical SNP position,” or “critical SNP locus” are useful to indicate whether or not a person with the particular critical SNP nucleotide is likely to be at either a “normal” risk or at a “high” risk, i.e., “greater than normal” risk of developing MRSA when exposed to the MRSA bacteria.

When the critical SNP nucleotide is present in the “high risk” or “MRSA susceptible form,” that individual has a much greater likelihood of developing a MRSA infection if exposed to a MRSA bacteria or infectious agent. Here this is also referred to as having a “high risk” or a “greater than normal risk” of developing a MRSA infection after exposure to the MRSA bacteria. In contrast to the “high risk” critical SNP nucleotides there are also the “normal risk” critical SNP nucleotide that a “normal” or “control” person has. A “normal” or “control” person is a person who has the “normal” or “control” version of the critical SNP nucleotides at the critical SNP position. The normal or control person is not associated with a greater likelihood, or high risk of contracting a MRSA infection after exposure to MRSA bacteria or SSI exposure. Thus the critical SNP position is the position or locus in the genome that holds a critical SNP nucleotide that is an important factor in determining whether or not an individual will have a normal or greater than normal risk of developing a MRSA infection if that individual is exposed to a MRSA bacteria. The critical SNP nucleotide and the information it provides can be combined with traditional risk factors to create an “overall MRSA risk profile” for any given individual.

These genetic markers are used in various combinations and in various ways with different preventive or treatment plans that can be fashioned and used to make their use appropriate and important for the control and treatment of possibly lethal bacterial infections. Each single nucleotide polymorphism (SNP) or “marker” and its location, or critical SNP position is identified and described. Their use and how to find and measure them and then confirm whether or not a person has one or more of these critical SNPs, or indicator SNPs, and their critical SNP location, and their critical SNP nucleotides, are described. These markers provide a means to reveal hidden signals in people that can be used to identify and proactively treat and prevent infection in people who may be at a high risk of developing MRSA or SSI, even those who have few or none of the typical risk factors. These markers or critical SNP positions and critical SNP nucleotides can also provide a means to reveal hidden signals in people that can be used to identify those people who might otherwise be incorrectly identified as needing proactive prevention or even treatment but who in fact are not at greater risk than “controls” or normal people. Methods, procedures, artificial probes and methods and procedures to use those probes are disclosed.

### DETAILED DESCRIPTION OF THE INVENTION

It should be understood that the invention is not limited to the particular embodiments of the invention described below, as variations of the particular embodiments would be known to one skilled in the art after reading this description. It is also to be understood that the terminology employed is for the purpose of describing particular embodiments, and is not intended to be limiting.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

### Definitions

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well-known and commonly employed in the art. Although any methods, devices and materials similar or equivalent to those described herein can be used in the practice or testing of the invention, the preferred methods, devices and materials are now described.

In this specification and the claims, the singular forms “a,” “an” and “the” include plural reference unless the context clearly dictates otherwise.

The practice of the present invention will employ, unless otherwise indicated, conventional techniques of molecular biology, microbiology, recombinant DNA technology, and immunology, which are within the skill of the art. Such techniques are explained fully in the literature. See, e.g., Sambrook et al. (2001) Molecular Cloning: A Laboratory Manual, 3rd ed., Cold Spring Harbor Press; DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

When a nucleotide sequence or base is a critical, then a “critical SNP position” is provided and the nucleotide base in the critical SNP position or locus is considered to be a critical SNP nucleotide or critical SNP nucleotide polymorphism. The knowledge of the critical SNP position and its critical SNP nucleotide polymorphism is but one factor in indicating or predicting whether the person with that critical SNP nucleotide has either a “normal risk” or a “high risk” of becoming infected with MRSA, should the person be exposed to MRSA bacteria. In this document a person with a “normal risk” is the same as a “control” or “control risk” person. In this document a person with a “greater than normal risk” of developing MRSA, is the same as a person with a “high,” “high risk” or “higher than normal risk” of becoming infected with MRSA if that person is exposed to MRSA bacteria. The critical SNPs have either control or normal critical SNP nucleotides which indicate the individual with that particular SNP being identified is consider “normal risk” individual with a normal risk of becoming infected with an antibiotic resistant Skin Infections, such as an antibiotic resistant Staph Infection or a MRSA infection, or an SSI infection, which indicates that should a person be exposed to such an antibiotic resistant infective agent he or she is likely to become infected with that antibiotic resistant agent. In this document a person is either at a “normal”, “control”, or “average” risk or the person is at “high” risk, “great” risk, or “greater than normal” risk of becoming infected with that antibiotic resistant agent, such as antibiotic resistant Staph Infection, MRSA or SSI, following exposure to the antibiotic resistant agent. In the “high”, “great” or “greater than normal risk” group the person will have one, more than one, or sometimes all 13 high risk SNPs, that is a “high risk variant in the genetic location that we refer to as the critical SNP nucleotide, or high risk critical SNP nucleotide polymorphism which indicates the particular SNP being identified is associated with a person having a high or greater than normal risk of becoming infected with an antibiotic resistant agent that promotes skin disease, such as antibiotic resistant Staph, MRSA or SSI. “Greater than normal” risk” herein is the same as “high” risk and “greater” risk of getting an antibiotic resistant infection following exposure to an antibiotic resistant agent.

The SNPs, either normal or high risk, are identified both by their associated genes, as well as their position or locus either in or in a described position relative to a described gene or genes. In at least one example between two defined and known genes of the human genome.

We clearly identify and precisely describe the location in the genome where there are special SNPs, or critical SNP positions, and what that nucleotide is in a normal person or a high risk person and whether that person has a normal or high risk of getting an antibiotic resistant Skin Infection such as a MRSA infection after exposure to a MRSA bacteria, or a Staph or SSI infection. We can also identify the person with a “normal” risk of getting a an antibiotic resistant Skin Infection such as a MRSA infection after exposure to a MRSA bacteria, or a Staph or SSI infection, this type of person may also be referred to as a “control”, “average” or a normal individual with a normal or average risk of getting a MRSA infection after exposure to antibiotic resistant Skin Infection. Sometimes identifying the normal or low risk individual can be as or more important to the patient and the treating physician as identifying the high risk individual.

The importance of being able to identify and treat the high risk individual cannot be overstated. We estimate that roughly 50 of every 300 individuals who are considered MRSA carriers who have had one or more MRSA infection may be considered as “high risk” individuals who are found to routinely carry the MRSA bacteria, and of those 50 we estimate that about 5 or 10 percent of those high risk individuals who carry the MRSA bacteria have a very high chance of dying from a MRSA infection within 2 years of their last infection, if they are not under a careful health watch.

In these descriptions and examples a single nucleotide base is critical to the identification and prediction of MRSA susceptibility and whether or not an individual patient has a high risk or a normal risk for developing a MRSA, HA-MRSA or CA-MRSA infection. In some situations, like that just described, the terminology used here is precise and descriptions are given for unique and critical SNP nucleotides for the purpose of describing particular embodiments of the invention.

Conversely, in other descriptions, such as with whole and partial genes, gene sequences, general oligonucleotide descriptions, artificial segments and such, it should be understood that this invention is not limited to specific individual or particular nucleotides, polymorphisms or oligonucleotide sequences, or polypeptide sequences. Sometimes such sequences can vary and such variations would be expected and understood by one of ordinary skill in the art. As we discuss herein, a typical gene has thousands of SNPs most of which have little to no known effects on the person with those SNPs, and we are not teaching or even suggesting that every nucleotide position is critical. For example the SEQ ID NOs provided in the specification and in the sequence listing that describe most of the gene sequences and that are not specifically said to be in a critical SNP positions, can, for the most part, have variations. It is only those SNPs in a critical SNP position, with a critical SNP nucleotide, or polymorphism associated with the described specific alpha numeric number (e.g., 1a, 2b, 3c, etc.), and in most cases have an “rs” number, that indicates the described nucleotides are limited to the precise DNA nucleotide described.

For example, nucleotides used in probes that are taught by these procedures and other procedures commonly known to one skilled in the art, may have numerous differences from the sequences we provide, both as to the full length gene and to partial gene sequences. A probe that is modified and used to detect the critical SNPs positions and their nucleotides may have some variation is its gene sequence from the sequences provided here and in the sequence listing.

Generally the longer the nucleotide sequence of the probe, the more variability is allowed for nucleotide substitutions that can still give the probe highly stringent hybridization but have little or minimum effect on the effectiveness of the probe.

In describing the present invention, the following terms will be employed and are intended to be defined as indicated below.

The expression “aggressive treatment” or “aggressive antibiotic resistant treatment” of these infections as well as methods of reducing or preventing recurrence, or prophylactic treatment of antibiotic resistant infection may include the following:

Decolonization, which tries to eliminate or at least reduce the bacteria from the skin and nasal passages by use of a variety of agents which can include: intranasal mupirocin, intranasal provodone iodine, intranasal alcohol and/or topical preparations for the skin including chlorhexidine or provodone iodine delivered via methods which include bath or impregnated cloths, or dilute bleach treatment via bath or impregnated cloths. In one embodiment, a dermal peel is used as an aggressive treatment.

Aggressive antibiotic therapy which raises the level of antibiotics used to more active antibiotics than normally used first-line which may include daptomycin, linezolid, tedezolid, or ceftaroline as well as combinations of antibiotics which could include the antibiotic rifampin which is synergistic with many other antibiotics.

Recolonization therapy can be used following decolonization which is an attempt to replace the “bad” Staph aureus bacteria with other bacteria considered non-pathogenic or “harmless” such as Staph epidermidis a typical normal bacteria found on the skin. This would be via lotions or other carriers containing live cultures of the bacteria.

In addition in cases where the patients are found to continue to recur, decolonization of family members, close contacts, or pets may be attempted as well as extensive environmental cleaning to remove reservoirs of Staph aureus in the local environment of the patient.

The expression “anti-MRSA antibiotics” refers to antibiotics that one ordinarily skilled in the art would understand to be antibiotics usually used to treat antibiotic resistant infections like MRSA. Examples of such drugs include Vancomycin, Daptomycin, Linezolid, Ceftaroline, and Telavancin, among other antibiotics.

The term, “biological sample” means any material or fluid (blood, lymph, etc.) derived from the body of a subject, that contains or may contain genomic DNA (chromosomal and mitochondrial DNA) or other oligonucleotides such as, for example, mRNA that derive from genomic DNA. Also included within the meaning of the term “biological sample” is an organ or tissue extract and culture fluid in which any cells or tissue preparation from a subject has been incubated. Methods of obtaining biological samples and methods of obtaining oligonucleotide molecules such as DNA and RNA from a biological sample are well known in the art.

The terms “complementary” or “complementarity” are used in reference to oligonucleotides related by the base-pairing rules for DNA-DNA, RNA-DNA and RNA-RNA pairing. For example, for the sequence “A-G-T,” is complementary to the sequence “T-C-A.” Complementarity may be “partial,” in which only some of the nucleic acid base pairs are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Under “low stringency” conditions, strands with a lower degree of complementarity will hybridize with each other. Under “high stringency conditions,” only strands with a higher degree of complementarity will remain hybridized with each other.

“Complementary” may be modified, as in the term “completely complementary,” refers to an oligonucleotide where all of the nucleotides are complementary to a target sequence (e.g., a miRNA). A completely complementary oligonucleotide may be shorter than the target sequence, thus, only hybridizing to a portion of the target.

“Complementary” may be modified, as in the term “partially complementary” refers to an oligonucleotide where at least one nucleotide is not complementary to (i.e., one or more “mismatches” with) the target sequence. Preferred partially complementary oligonucleotides are those that can still hybridize to a target sequence under physiological conditions. A particular partially complementary oligonucleotide may have a ‘random’ pattern of one or more mismatches with the target sequence throughout the oligonucleotide (although the pattern of mismatches is preferentially constrained by retention of the ability to still hybridize to the target sequence under physiological conditions). A particular partially complementary oligonucleotide may have regions where the oligonucleotide sequence is highly, or even completely complementary to a target sequence, and regions where the oligonucleotide is not complementary, or is less complementary to the target sequence.

“Complementary” is illustrated, for example, partially complimentary oligonucleotides may have one or more regions that hybridize to a target sequence, and one or more regions that do not hybridize to the target sequence. Thus, a partially complementary sequence (such as a PCR or reverse transcriptase (RT) primer) may hybridize to a portion (i.e., the middle, the 5′, or 3′ end) of a particular target sequence, and not hybridize with the rest of the target sequence. Oligonucleotides with mismatches at the ends may still hybridize to the target sequence. Partially complementary sequences may be capable of binding to a sequence having less than 60%, 70%, 80%, 90%, 95%, to less than 100% identity to the target sequence. For purposes of defining or categorizing partially complementary sequences, a partially complementary sequence or region of a sequence becomes more complementary or becomes “highly complementary” as it approaches 100% complementarity to a target sequence. Thus, a highly complementary sequence may have 60%, 70%, 80%, 90%, 95%, to 99% identity to all or a portion of a target sequence. The exact percentage identity of the highly complementary sequence may depend on the length of the highly complementary sequence and the desired stringency and specificity of hybridization. Partially complementary sequences may hybridize to one or more target sequences. As we note, partially complementary sequences may be completely complementary or highly complementary to a portion of the target sequence, such that they are completely or highly complementary to, e.g., 5%, 10%, 20%, 30%, 40%, 50% 60%, 70%, 80%, 90%, 95%, 99% of the target sequence. Similarly, 5%, 10%, 20%, 30%, 40%, 50% 60%, 70%, 80%, 90%, 95%, 99% of the partially complementary sequence may be completely complementary or highly complementary to all or a portion of the target sequence.

By the term “control” or “normal” we mean a person who is of typical or average susceptibility to a MRSA infection after suffering from a MRSA exposure. A person or nucleotide that is considered a “normal” or a “control” in this description, is one with an average risk of developing a MRSA infection after exposure to MRSA. This normal or control person or nucleotide is to be contrasted with a “MRSA high risk” person who has a greater than normal risk of developing a MRSA infection after exposure to MRSA than would a normal or control person. The risk the MRSA high risk person faces is least twice as likely, (2×), to develop a MRSA infection following exposure to MRSA than the risk a normal or control person would have to develop a MRSA infection after they are exposed to MRSA. The control person has at least half or less chance of developing a MRSA infection after they are exposed to MRSA than a high risk person would have, and often a much higher chance of getting a MRSA infection.

The expression “critical SNP position” indicates the location or position, in or between the indicated gene or genes where the indicated SNP has the critical nucleotide that is an indicator of whether the person, patient or subject has a normal or higher than normal risk of developing a MRSA or SSI infection following exposure to the bacteria. The critical SNP nucleotide is indicative of a person being either “normal/control” or indicative of a person who has “greater than normal/high” risk of MRSA or SSI infection following exposure to MRSA.

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Decolonization” is usually in reference to decolonization of Staph aureus and usually tries to eliminate or at least reduce the bacteria from the skin and nasal passages as well as the throat perineum and rectum. A variety of agents, both topical and systemic, have been used to decolonize S. aureus. Mupirocin (Bactroban Nasal made by GlaxoSmith Kline or Mupirocin nasal ointment is commonly used. Rifampin and doxycycline are sometimes used. Other treatments involve body washing with chlorhexidine. Sometimes body washing is applied with antibiotic treatments. Sometimes attempts to decolonize an individual have poor success rates when follow up cultures are taken of nasal flares, perineum, skin, and medical device exit sites.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified gene polymorphic sequences. Thus, “detectable moiety” is used synonymously with “label molecule.” Synthesis of oligonucleotides will include the critical SNP at the critical SNP position can be accomplished by any one of several methods known to those skilled in the art. Label moieties or molecules are known to those skilled in the art as being useful for detection of the target and they include: radioactive, chemiluminescent, fluorescent and luminescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

A “diagnosis” of MRSA, HA or CA-MRSA (here usually just MRSA) may include the early detection of the disease or a confirmation of a diagnosis of the disease that has been made from other signs and/or symptoms. A “diagnosis” can include a diagnosis of increased risk or high risk of development or recurrence of MRSA. A diagnosis may include a “prognosis,” that is, a future prediction of the progression of MRSA based on the presence or absence of one or more SNPs associated with MRSA or MRSA. A diagnosis or prognosis may be based on one or more samplings of DNA or RNA from a biological sample obtained from a subject. An “increased risk” of developing MRSA or MRSA may be diagnosed by the presence of one or more SNPs characteristic of a phenotype of susceptibility to recurrent MRSA in otherwise asymptomatic or undiagnosed subjects.

“DNA amplification” as used herein refers to any process that increases the number of copies of a specific DNA sequence by enzymatically amplifying the nucleic acid sequence. A variety of processes are known. One of the most commonly used is polymerase chain reaction (PCR). PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, real-time PCR, etc., may be used. In general, the PCR amplification process involves a cyclic enzymatic chain reaction for preparing exponential quantities of a specific nucleic acid sequence. It requires a small amount of a sequence to initiate the chain reaction and oligonucleotide primers that will hybridize to the sequence. In PCR, the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

A DNA “coding sequence” or a “nucleotide sequence encoding” a particular protein is a DNA sequence that is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. “Non-coding” genomic sequences may include regulatory, RNA transcription sequences (rRNA, tRNA, miRNA, etc.), introns and other non-gene sequences, such as structural sequences, putatively non-functional sequences (“junk DNA”) and the like.

The terms “enzymatically amplify”, “enzymatically amplifying”, “amplify” and “amplifying” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Qβ-replicase amplification (QβRA); self-sustained replication (3 SR); and NASBA (nucleic acid sequence-based amplification), which can be performed on RNA or DNA as the nucleic acid sequence to be amplified.

A “fragment” of a molecule such as a protein or nucleic acid is meant to refer to a portion of a longer or larger amino acid or nucleotide genetic sequence.

The term “genome” refers to all the genetic material in the chromosomes of a particular organism. Its size is generally given as its total number of base pairs. Within the genome, the term “gene” refers to a locatable region of genomic sequence, corresponding to a unit of inheritance, which is associated with regulatory regions, transcribed regions and/or other functional sequence regions. “Non-gene regions” of the genome have or appear to have no functional role, but may have a structural (e.g., regions near the centromere) or unknown regulatory function. The physical development and phenotype of organisms can be thought of as a product of genes interacting with each other and with the environment. A concise definition of “gene” taking into account complex patterns of regulation and transcription, sequence conservation and non-coding RNA genes has been proposed by Gerstein et al. (Genome Research 17 (6), 669-681, 2007) “A gene is a union of genomic sequences encoding a coherent set of potentially overlapping functional products.” Id. In general, an individual's genetic characteristics, as defined by the nucleotide sequence of its genome, are known as its “genotype,” while an individual's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

By “hybridization” or “hybridizing,” as used herein, is meant the formation of A-T and C-G base pairs between the nucleotide sequence of a fragment of a segment of a oligonucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and a sensor probe. It may also include an anchor probe.

In a hybridization complex, two nucleic acid fragments are considered to be “selectively hybridizable” to a oligonucleotide if they are capable of specifically hybridizing to a nucleic acid or a variant thereof or specifically priming a polymerase chain reaction: (i) under typical hybridization and wash conditions, as described, for example, in Sambrook et al. supra and Nucleic Acid Hybridization, supra, (ii) using reduced stringency wash conditions that allow at most about 25-30% base pair mismatches, for example: 2×SSC, 0.1% SDS, room temperature twice, 30 minutes each; then 2×SSC, 0.1% SDS, 37° C. once, 30 minutes; then 2×SSC room temperature twice, 10 minutes each, or (iii) selecting primers for use in typical polymerase chain reactions (PCR) under standard conditions (described for example, in Saiki, et al. (1988) Science 239:487-491).

In a hybridization complex, the term “capable of hybridizing under stringent conditions” refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of a nucleic acid or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g., low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° Celsius in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

A hybridization complex may refer to “stringent conditions” which typically will be those in which the salt concentration is less than about 1.5 M sodium ion, typically about 0.01 to 1.0 M Na ion concentration (or other salts) from about pH 7.0 to about pH 8.3 and the temperature is at least about 30° Celsius for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1-2×SSC at 50 to 55° C. Exemplary moderate stringency conditions include hybridization in 40 to 45% formamide, 1 M NaCl, 1% SDS at 37° C., and a wash in 0.5-1×SSC at 55 to 60° C. Exemplary high stringency conditions include hybridization in 50% formamide, 1 M NaCl, 1% SDS at 37° C., and a wash in 0.1×SSC at 60 to 65° C.

An “isolated” oligonucleotide or polypeptide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

As used herein, the term “locus” or “loci” refers to the location of a coding, regulatory or non-coding region on a chromosome. Absolute location of a region may be known to more or less precision (i.e., a locus is known to be located within a chromosome, chromosome arm, chromosome band, or to the nearest kilobase or base pair of a chromosome) due to the fact that genome length may differ slightly between individuals or the precise location of a locus is not known. A relative location may also be specified where a locus is located within a sequenced fragment of a chromosome. Pairs of genes, known as “alleles” may be present for a particular locus in organisms, such as humans, that are diploid (usually contain two copies of most chromosomes) in most cells and tissues. An individual's particular combination of alleles is referred to as its “genotype”. Where both alleles are identical the individual is said to be homozygous for the trait controlled by that gene pair; where the alleles are different, the individual is said to be heterozygous for the trait. While inclusive of loci within coding regions, an “allele” may also be present at locations in non-coding regions. Certain organisms, cells or tissues may be haploid or polyploid (triploid, etc.) and have more or less than two alleles at a particular locus.

A “melting temperature” is meant the temperature at which hybridized oligonucleotide duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1° C. to about 10° C. so as to be readily detectable.

MRSA is a strain of S. aureus that has become resistant to methicillin, penicillin, amoxicillin and oxacillin among other antibiotics that are commonly used to treat ordinary S. aureus infections. When MRSA is acquired or develops in health care or medical care facilities, such as hospitals, it is known as hospital acquired or health care-associated MRSA. When MRSA develops in otherwise healthy people not exposed to hospital situations it is termed community-acquired MRSA. Here it should be understood that MRSA includes both HA-MRSA and CA-MRSA types of MRSA. Some people and animals are carriers of MRSA, including dogs and cats, and while they may carry MRSA, some carriers never develop MRSA infections, some do, and some people who are exposed to MRSA are at much greater risk than others of developing a MRSA infection. Sometimes a veterinary office will become a location where MRSA is easily spread. Sometimes an NFL locker room will become a location where MRSA is easily spread.

The term “normal” or “control” person means a person who has a typical or average susceptibility to getting a MRSA infection after being exposed to MRSA bacteria. A normal or control person typically has at least half the chance or risk of developing a MRSA infection as a person of increased or high risk of MRSA infection. Sometimes the high risk person will have as much as 10 times or even greater risk of having a MRSA infection than a normal person has of getting a MRSA infection even when the “normal” person and the “high risk” person have the same exposure to the MRSA bacteria.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). An “isolated” nucleic acid molecule is one that is separated from other nucleic acid molecules that are present in the natural source of the nucleic acid.

A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The term “oligonucleotide” refers to a series of linked nucleotide residues. The series of nucleotide residues are connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides linked by a phosphodiester backbone. Oligonucleotides may be used, for example, as primers in a PCR reaction, or as probes to detect the presence of a certain sequence in or within a nucleic acid molecule. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. A “modified oligonucleotide” refers to an oligonucleotide in which one or more natural nucleotides have been partially, substantially, or completely replaced with modified nucleotides.

The term “oligonucleotide encoding a peptide or protein” as used herein refers to a DNA fragment or isolated DNA molecule encoding a protein, or the complementary strand thereto; but, RNA is not excluded, as it is understood in the art that thymidine (T) in a DNA sequence is considered equal to uracil (U) in an RNA sequence. Thus, RNA sequences for use in the invention, e.g., for use in RNA vectors, can be derived from DNA sequences, by thymidine (T) in the DNA sequence being considered equal to uracil (U) in RNA sequences.

The following are non-limiting examples of oligonucleotides: a gene or gene fragment, exons, introns, mRNA, tRNA, rRNA, ribozymes, cDNA, recombinant oligonucleotides, branched oligonucleotides, plasmids, vectors, isolated DNA of any sequence, isolated RNA of any sequence, nucleic acid probes and primers. An oligonucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the oligonucleotide to proteins, metal ions, labeling components, other oligonucleotides or solid support.

The expression “overall MRSA risk profile” includes many factors or elements including but not only whether or not a person has a “critical SNP nucleotide” that indicates high MRSA risk factor but also includes various other factors such as whether or not the person has traditional MRSA risk factors, examples are living in crowded unsanitary conditions, participating in sports or other activities involving numerous and repeated cuts and injuries and other known general MRSA risk factors.

“Percent identity” can be determined by hybridization of oligonucleotides under conditions that form stable duplexes between similar regions, followed by digestion with single-stranded-specific nuclease(s), and size determination of the digested fragments. DNA sequences that are homologous can be identified in a Southern hybridization experiment under, for example, stringent conditions, as defined for that particular system. Defining appropriate hybridization conditions is within the skill of the art. See, e.g., Sambrook et al. supra; DNA Cloning, supra; Nucleic Acid Hybridization, supra.

PDT is Persistent and Difficult to Treat. An SSI may be one that is a PDT SSI.

A “polymerase” is an enzyme that catalyzes the sequential addition of monomeric units to a polymeric chain, or links two or more monomeric units to initiate a polymeric chain. The “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of an oligonucleotide chain in a template-dependent manner, thereby synthesizing a nucleic acid that is complementary to the template molecule. Polymerases may be used either to extend a primer once or repetitively or to amplify an oligonucleotide by repetitive priming of two complementary strands using two primers. A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

A “primer” is an oligonucleotide, the sequence of at least of portion of which is complementary to a segment of a template DNA which is to be amplified or replicated. Typically primers are used in performing the polymerase chain reaction (PCR). A primer hybridizes with (or “anneals” to) the template DNA and is used by the polymerase enzyme as the starting point for the replication/amplification process. The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety. The probes may vary in length from about, any of the following and any range of numbers that includes any and all of the following numbers: 5, 10, 20, 30, 40, an 50, sequences in length for the typical relatively short probes to and to 60, 70, 80, 90 or 100 sequences in length for longer probes, in rare situations probes of up to medium length probes to 200, 300, 400, 500, 600, 700, 800, 900, and 1000, bases in length could be used for very long probes.

A “resistant skin infection” is a skin infection that does not respond to nor becomes eliminated after routine treatment with antibiotics including methicillin, penicillin, amoxicillin, oxacillin and other antibiotics used to treat common skin infections. A “resistant skin infection” can also mean an infection that at first appears to be successfully treated with commonly used antibiotics but then reappears, usually with within 1 to 3 months, and without any apparent reinfection from an infectious agent. Resistant skin infections may be caused by the properties of the infectious agents, being highly resistant to antibiotics and or they can result from common forms of infectious agents that reside on individuals who have abnormal or weaker than normal abilities to resist common skin infections.

A “restriction fragment” refers to a fragment of an oligonucleotide generated by a restriction endonuclease (an enzyme that cleaves phosphodiester bonds within an oligonucleotide chain) that cleaves DNA in response to a recognition site on the DNA. The recognition site (restriction site) consists of a specific sequence of nucleotides typically about 4-8 nucleotides long.

“Sequence identity” refers to the percent identity between two oligonucleotides or two polypeptide moieties. Genes that share a high sequence identity or similarity support the hypothesis that they share a common ancestor and are therefore homologous. Sequence homology may also indicate common function. Two DNA, or two polypeptide sequences are similar to each other and may be homologous when the sequences exhibit at least about 80%, 81%, 82%, 83%, 84%, 85%, 86%, 87%, 88%, 89%, preferably at least about 90%, 91%, 92%, 93%, 94% and most preferably at least about 95%, 96%, 97%, 98%, 99%, 99.5%, 99.9% sequence identity over a defined length of the molecules. As used herein, sequence identity also refers to sequences showing complete identity (100% sequence identity) to the specified DNA or polypeptide sequence.

“Skin infections,” means any infection caused by any of the following: Staph infections, MRSA infections, SSI, or any other infection that involves the skin and or that extends to other structures within the skin, and especially rupture or penetration of the skin. A skin infection includes the infections listed under the definitions of MRSA and SSI infections. For “resistant skin infections,” see definition above.

SSI can appear as an abscess, infection or inflammation of the skin often associated with a wound and sometimes a surgical would. An SSI can be, but is not always, caused by a MRSA infection. An SSI is associated with a surgical incision usually caused by surgery. An SSI is an infection of the skin near the site of a wound to the skin. Of special interest here are SSIs that are persistent and difficult to treat, known herein as PDT SSIs.

“Staph” refers to the genus of bacteria called Staphylococcus. Staph infections sometimes cause boils, furuncles, impetigo, abscesses, food poisoning, mastitis, pneumonia, osteomyelitis, endocarditis, bacteremia, sepsis, cellulitis and toxic shock syndrome. All of these and related diseases may be affected by the disclosures here. One species of Staph is Staphylococcus aureus. MRSA are Staphylococcus aureus that have become resistant to antibiotics including methicillin, penicillin, amoxicillin, and oxacillin.

A “template” refers to a target oligonucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such a DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional oligonucleotides.

A “thermocyclic reaction” is a multi-step reaction wherein at least two steps are accomplished by changing the temperature of the reaction.

A “variance” is a difference in the nucleotide sequence among related oligonucleotides. The difference may be the deletion of one or more nucleotides from the sequence of one oligonucleotide compared to the sequence of a related oligonucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same oligonucleotide.

A “single nucleotide polymorphism” or “SNP” refers to a variation in the nucleotide sequence of an oligonucleotide that differs from another related oligonucleotide by a single nucleotide difference. For example, without limitation, exchanging one A for one C, G or T in the entire sequence of oligonucleotide constitutes a SNP. It is possible to have more than one SNP in a particular oligonucleotide. For example, at one position in an oligonucleotide, a C may be exchanged for a T, at another position a G may be exchanged for an A and so on. When referring to SNPs, the oligonucleotide is most often DNA. SNPs can be found in coding regions of the genome (i.e., within an exon) or non-coding intragenic (i.e., in an intron) or intergenic regions. In this application we have identified 13 special SNPs and they may be referred to by their position relative to the other nucleotides in the gene they are in or near, as a SNP that is either normal or one that indicates a greater risk of developing MRSA infection after exposure to MRSA and they may be identified by their relative position or locus, or their “critical SNP position”, that is where in the gene or probe oligonucleotide they are known to exist.

RNA sequences within the scope of the invention are derived from the DNA sequences, by thymidine (T) in the DNA sequence being considered equal to uracil (U) in RNA sequences.

“Subject” or “Patient” as used herein refers to a mammal, preferably a human, in need of diagnosis and/or treatment for a condition, disorder or disease.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

Brief Description of the Sequences Listed in the Specification

SEQ ID NO 1 is the full sequence of the Fam129B gene which shows the MRSA SNP, an A or adenine, at position 17460, indicating those with a high risk of a MRSA or SSI infection. The sequence contains 73,651 nucleotides.

SEQ ID NO 2 is the full sequence of the Fam129B gene which shows the control SNP, a C or cytosine, at position 17460, indicating those at normal risk of a MRSA or SSI infection. The sequence contains 73,651 nucleotides.

SEQ ID NO 3 is a partial sequence of the Fam129B gene which shows an A or adenine at the 17^{th} position in the partial gene sequence for those with a high risk of a MRSA or SSI infection. The sequence contains 33 nucleotides.

SEQ ID NO 4 is a partial sequence of the Fam129B gene which shows a C or cytosine at the 17^{th} position in the partial gene sequence for those at normal risk of a MRSA or SSI infection. The sequence contains 33 nucleotides.

SEQ ID NO 5 is the full gene sequence of the RBM6 gene which shows that at position 26162, the variable “n” can be either C (cytosine) or G (guanine). C is high risk and G is normal. The sequence contains 137,212 nucleotides.

SEQ ID NO 6 is a partial gene sequence of the RBM6 gene which shows a C or cytosine at the 26^{th} position in the partial gene sequence for those with a high risk of MRSA or SSI (control). The sequence contains 51 nucleotides.

SEQ ID NO 7 is a partial gene sequence of the RBM6 gene which shows a G or guanine at the 26^{th} position in the partial gene sequence for those at normal risk of MRSA or SSI (control). The sequence contains 51 nucleotides.

SEQ ID NO 8 is the full gene sequence of the KANK 4 gene which shows at position 76547, the variable “n” can be either G (guanine) or T (thymine). G is high risk and T is normal. The sequence contains 83,248 nucleotides.

SEQ ID NO 9 is a partial gene sequence of the KANK 4 gene which shows a G or guanine at the 26^{th} position in the partial gene sequence for those with a high risk of developing MRSA or SSI. The sequence contains 51 nucleotides.

SEQ ID NO 10 is a partial gene sequence of the KANK 4 gene which shows a T or thymine at the 26^{th} position in the partial gene sequence for those at normal risk of developing MRSA or SSI. The sequence contains 51 nucleotides.

SEQ ID NO 11 is the full gene sequence of the uncharacterized protein C10orf35 isoform X1 gene, plus additional nucleotides beyond the C10orf35 isoform X1 gene up to and including 500 nucleotides beyond SNP/rs2642572. SEQ ID NO 11 terminates before the beginning of the Col 13A1 gene. SEQ ID NO 11 has the nucleotides for the C10orf35 isoform XI as the first 3,353 nucleotides, followed by 155,813 nucleotides before the SNP, the SNP is at position 159,166 of SEQ ID NO 11 and then there are 500 more nucleotides in this sequence. It shows that at position 159166, the variable “n” can be either G (guanine) or A (adenine). G is high risk and A is normal. The sequence contains 159,666 nucleotides.

SEQ ID NO 12 is a partial gene sequence of SEQ ID NO 11 which shows a G or guanine at the 26^{th} position in the partial gene sequence for those with a high risk of developing MRSA or SSI. The sequence contains 51 nucleotides.

SEQ ID NO 13 is a partial gene sequence of SEQ ID NO 11 which shows an A or adenine at the 26^{th} position in the partial gene sequence for those at normal risk of developing MRSA or SSI. The sequence contains 51 nucleotides.

SEQ ID NO 14 is the full gene sequence of the Col 19A1 gene which shows that 1) at position 151755, the variable “n” can be either A (adenine) or G (guanine); 2) at position 152225, N can be either T (thymine) or C (cytosine); 3) at position 162547, N can be either G (guanine) or A (adenine); 4) at position 162830, N can be either G (guanine) or A (adenine); and 5) at position 163341, N can be either T (thymine) or G (guanine). The sequence contains 345,899 nucleotides.

SEQ ID NO 15 is a partial gene sequence of the Col 19A1 gene which shows a T or thymine at the 21^{st} position in the partial gene sequence for those with a high risk of MRSA or SSI. The sequence contains 41 nucleotides.

SEQ ID NO 16 is a partial gene sequence of the Col 19A1 gene which shows a G or guanine at the 21^{st} position in the partial gene sequence for those at normal risk of MRSA or SSI. The sequence contains 41 nucleotides.

SEQ ID NO 17 is a partial gene sequence of the Col 19A1 gene which shows a G or guanine at the 26^{th} position in the partial gene sequence for those with a high risk of MRSA or SSI. The sequence contains 51 nucleotides.

SEQ ID NO 18 is a partial gene sequence of the Col 19A1 gene which shows an A or adenine at the 26^{th} position in the partial gene sequence for those at normal risk of MRSA or SSI. The sequence contains 51 nucleotides.

SEQ ID NO 19 is a partial gene sequence of the Col 19A1 gene which shows a G or guanine at the 26^{th} position in the partial gene sequence for those with a high risk of MRSA or SSI. The sequence contains 51 nucleotides.

SEQ ID NO 20 is a partial gene sequence of the Col 19A1 gene which shows an A or adenine at the 26^{th} position in the SNP for those at normal risk of MRSA or SSI. The sequence contains 51 nucleotides.

SEQ ID NO 21 is a partial gene sequence of the Col 19A1 gene which shows a T or thymine at the 26^{th} position in the SNP for those with a high risk of MRSA or SSI. The sequence contains 51 nucleotides.

SEQ ID NO 22 is a partial gene sequence of the Col 19A1 gene which shows a C or cytosine at the 26^{th} position in the partial gene sequence for those at normal risk of MRSA or SSI. The sequence contains 51 nucleotides.

SEQ ID NO 23 is a partial gene sequence of the Col 19A1 gene which shows an A or adenine at the 26^{th} position in the partial gene sequence for those with a high risk of MRSA or SSI. The sequence contains 51 nucleotides.

SEQ ID NO 24 is a partial gene sequence of the Col 19A1 gene which shows a G or guanine at the 26^{th} position in the partial gene sequence for those at normal risk of MRSA or SSI. The sequence contains 51 nucleotides.

SEQ ID NO 25 is the full gene sequence of the IGSF8 gene which shows at position 6692, the variable “n” can be either G (guanine) or A (adenine). G is high risk and A is normal. The sequence contains 7,521 nucleotides.

SEQ ID NO 26 is a partial gene sequence of the IGSF8 gene which shows a G or guanine at the 26^{th} position in the partial gene sequence for those with a high risk of MRSA or SSI. The sequence contains 51 nucleotides.

SEQ ID NO 27 is a partial gene sequence of the IGSF8 gene which shows an A or adenine at the 26^{th} position in the partial gene sequence for those at normal risk of MRSA or SSI. The sequence contains 51 nucleotides.

SEQ ID NO 28 is the full gene sequence of the ADARB2 gene which shows at position 223202, the variable “n” can be either A (adenine) or G (guanine). A is high risk and G is normal. The sequence contains 560,186 nucleotides.

SEQ ID NO 29 is a partial gene sequence of the ADARB2 gene which shows an A or adenine at the 21^{st} position in the partial gene sequence for those with a high risk of MRSA or SSI. The sequence contains 41 nucleotides.

SEQ ID NO 30 is a partial gene sequence of the ADARB2 gene which shows a G or guanine at the 21^{st} position in the partial gene sequence for those at normal risk of MRSA or SSI. The sequence contains 41 nucleotides.

SEQ ID NO 31 is the full gene sequence of the DCT gene which shows at position 81032, the variable “n” can be either C (cytosine) or T (thymine). C is high risk and T is normal. The sequence contains 112,962 nucleotides.

SEQ ID NO 32 is a partial gene sequence of the DCT gene which shows a C or cytosine at the 22^{nd} position in the partial gene sequence for those with a high risk of MRSA or SSI. The sequence contains 43 nucleotides.

SEQ ID NO 33 is a partial gene sequence of the DCT gene which shows a T or thymine at the 22^{nd} position in the partial gene sequence for those at normal risk of MRSA or SSI. The sequence contains 43 nucleotides.

SEQ ID NO 34 is the full gene sequence of the CCR2 gene, Chr3: 46,353,744-46,360,940. The sequence contains 14,394 nucleotides.

SEQ ID NO 35 is the gene sequence which starts at position 46,360,941 (which is the first nucleotide after the end of the CCR2 gene) and ends at position 46,425,932; the SNP/rs4619820 is contained between these positions, after the CCR2 gene but before the LTF gene. This sequence includes all the nucleotides after the CCR2 gene, up to and including the SNP and an additional 500 nucleotides. The LTF gene downstream of this sequence is not included. The LTF gene is also in Chr 3, spanning Chr3: 46,436,005-46,465,142. The SNP is located at position 64492 of SEQ ID NO 35, the variable “n” can be either G (guanine) or A (adenine). G is high risk and A is normal. SEQ ID NO 35 contains 64,992 nucleotides.

SEQ ID NO 36 is a partial gene sequence of SEQ ID NO 35 which shows a G or guanine at the 26^{th} position in the partial gene sequence for those with a high risk of MRSA or SSI. The sequence contains 51 nucleotides.

SEQ ID NO 37 is a partial gene sequence of SEQ ID NO 35 which shows an A or adenine at the 26^{th} position in the partial gene sequence for those at normal risk of MRSA or SSI. The sequence contains 51 nucleotides.

In the sequence listing we provide the full length of natural gene sequences, and artificial fragments, probes both large and small, and fragments of various genetic regions that either include or bracket the single nucleotide polymorphisms, known as SNPs, also known as critical SNP nucleotides that have a locus at a critical SNP position, and they are an important component in identifying both individuals at high risk of developing a MRSA infection and individuals that are not at high risk of developing a MRSA infection but who might normally be placed in that category. We provide genetic sequences, some identical, some artificial, that bracket the site of these SNPs, or “critical SNP positions.” The precise sequences we provide are usually relatively short sequences that identify those unique critical nucleotides (SNPs) that we have discovered are useful to determine whether a person is one of what we say are two possible types of individuals. One type is what we refer to here as a “normal” also referred to herein as a “control” and sometimes as a “low risk” individual, this is a person who is NOT predisposed to develop repeated and multiple MRSA infections after a single exposure to the MRSA bacteria. Rather, a control, normal or low risk person has a typically low chance of developing a MRSA infection after exposure to MRSA. And more importantly when a control or typical person does have a MRSA infection, it can be treated and cured relatively easily and without relapse. The majority of people exposed to MRSA, once treated, do not repeatedly redevelop a MRSA infection. The majority of people are what we call normal, control or low risk and if are exposed MRSA, and get a MRSA infection and they get a standard treatment, usually with antibiotics, they recover and they may never have a similar infection again. The normal or control is compared and contrasted with what we call here a high risk, high risk, or abnormal individual.

Evaluating the MRSA and SSI High Risk Patient

We describe methods of evaluating patients to determine if they are at increased risk of developing an infection from MRSA or SSI. Even patients who have no previously known risk factors or observable indications, symptoms or complaints of an infection and without any history of being resistant to MRSA or SSI may be at high risk of developing a MRSA infection. Also important we describe how to identify those patients who might have some or all of the risk factors that would ordinarily put a patient on the high risk watch and prevent list but who in fact is not at high risk of developing a MRSA or SSI infection. Using the procedures, tests and materials described herein, a caregiver will be able to better predict and assess what an individual patient's true level of risk really is, and then act to accordingly, either to prevent a risk that is there, or in some cases, to save the resources that would likely be wasted to prevent a risk that is actually a normal risk but falsely seems high because of the standard risk factors. Using the steps and procedures described herein health care providers can better determine when certain steps should be taken or administered to prevent patients from becoming infected with MRSA and SSI, even though the patient may have none of the previously known risk factors of developing MRSA or SSI. We describe and claim these procedures, tests and materials.

Until now there have been no generally accepted methods to test an individual's susceptibility to getting a MRSA infection. Now we can describe how to find, test and use over a dozen unique nucleotide polymorphisms, which if properly sampled and evaluated, can lead to accurate predictions of which individuals are more likely to be sickened by a MRSA infection than are “control” or “normal” individuals that are not at a higher risk of developing the disease. As a consequence, the susceptible individuals that can now be identified can be given better care in order to better prevent susceptible individuals from becoming sick and thus prevent or reduce the spread of this sometimes deadly disease. We describe the various unique SNPs as well as unique combinations of these genetic polymorphisms or (Genetic Marker Combinations) and how they can be used to predict a patient's risk and to administer prophylactic treatment or preventative steps.

It should be understood that most SNPs in a human's gene do not indicate or reveal anything useful or special to a physician. For example, in one of the genes we discuss here, the KANK4 gene, it is estimated that there are 4,600 SNPs in the KANK4 gene alone. We believe only 1 of those 4600 SNPs, just one base nucleotide, is an indicator, or critical SNP or a risk factor for that person getting a MRSA or SSI infection following exposure to the MRSA bacteria. Knowing this SNP exists and how to use it and determining which SNP it is and where that SNP is in the genome and how it can be used as an indicator of MRSA susceptibility as to assist in treatment and in making the lives of individuals born with a predisposition of getting MRSA infections is just one important aspect of the invention and claims described here. Further, referring again to our KANK4 gene example, if only 1000 base pairs around the site of the SNP were analyzed (500 bases before and 500 after) one would find about 48 other SNPs in that 1000 base pair region, that had nothing to with MRSA or SSI susceptibility. This is the “noise” that hides the existence and position of these valuable SNPs. These SNPs and their critical SNP position and nucleotide can be thought of as hidden signals. The hidden signals can't be seen or heard and until now they couldn't be detected. They are revealed here and we teach how they can be used. And those averages of about 4,600 SNPs in the KANK4 gene alone are just the number of SNPs in just one single gene. Altogether the KANK4 gene has about ˜83 kbases in total. We suspect that ratio, about 4,800 SNPs for every 83,000 bases that have no relationship at all to MRSA or SSI susceptibility, is an average ratio of SNP per base pair for all the genes we describe here. This is the “noise” ratio. We did not examine the other genes presented here to precisely determine the total number of SNPs, but we believe, to a scientific certainty, that the other genes discussed here would most likely have an equally high level of total SNPs present if the size of the other genes was similar to the size of the KANK4 gene. In this case, neither the SNPs nor the genes where the SNPs are located (either in or near) have been previously identified as holding a SNP that indicates a greater susceptibility to a MRSA infection. We teach how these previously secret and unknown SNPs can be heard and the individuals who have them identified, over and in spite of all the SNP noise in a genome, and then used for this one specific purpose in order to better prevent infections and save lives.

Once a susceptible patient has been identified, preventive steps can be taken such as placing people who are susceptible to MRSA or SSI in an environment that limits their exposure to MRSA such as isolation from others, and treatment with MRSA medications, including preventive or prophylactic treatments, in order to protect the patient and to prevent the spread of MRSA. When surgery is contemplated a person at high risk of SSI may be given special prophylactic treatment and medicines and appropriate palliative care. Contaminated surfaces and laundry items can be more thoroughly disinfected. People visiting or caring for susceptible people may be required to wear protective garments and follow strict hygiene procedures.

The invention is also of great importance in order to identify those who are NOT susceptible or at increased risk of developing MRSA or SSI. Use of these probes and materials can save time, money, effort and resources that could otherwise be wasted taking extreme measures protecting a patient and performing other preventive steps, when they are not needed, trying to prevent a disease that is not likely to develop even without preventive measures. It can be a large waste of time, money and effort to put a patient in isolation that doesn't need to be in isolation.

The SSI and MRSA Family of SNPs Related to Skin and Staph Infections

We describe a family of SNPs or genetic markers, that if present in an individual, either individually or in combination, indicates a greater than normal risk or greater than normal chance that the individual with the SNP or marker might develop MRSA or SSI if exposed to the bacteria that cause MRSA or SSI. Most of the SNPs we describe are within genes but two SNPs are between genes. The SNPs or markers we describe are in or near the following genes: FAM 129B (1 SNP), RBM6 (1 SNP), KANK 4 (1 SNP), Col 13A1-close (1 SNP), COL 19A1 (5 SNPs), IGSF8 (1 SNP), ADARB2 (1 SNP), DCT (1 SNP), and the last SNP lies between genes LTF and CCRL-2. The number in parenthesis is the number of SNPs associated with that gene.

The SNPs in this document are given a unique number and those SNP numbers are:

“a1” is known as SNP/rs2249861 and it is within the FAM 129B gene;
“b2” is known as SNP/rs2352969 and it is within the RBM6 gene;
“c3” is known as SNP/rs12758910 and it is within the KANK 4 gene;
“d4” is known as SNP/rs2642572 and it is outside and between two genes, the C10orf35 isoform X1 gene and the Col 13A1 gene;
“e5” is known as SNP/rs3793038 and it is within the Col 19A1 gene;
“f6” is known as SNP/rs3805987 and it is within the Col 19A1 gene;
“g7” is known as SNP/rs9454944 and it is within the Col 19A1 gene;
“h8” is known as SNP/rs978291 and it is within the Col 19A1 gene;
“i9” is known as SNP/rs978290 and it is within the Col 19A1 gene;
“j10” is known as SNP/rs1131891 and it is within the IGSF8 gene;
“k11” is known as SNP/rs127711411 and it is within the ADARB2 gene;
“l12” is known as SNP/rs4773794 and it is within the DCT gene; and
“m13” is known as SNP/rs4619820 and it is outside and between two genes, CCR2 and LTF.

Any single SNP or any combination of two or more of the above SNPs, when present in an individual and characterized as a MRSA high risk SNP, can function as a component of an indicator system (i.e., a critical high risk SNP) that can lead to the conclusion that the individual is at increased, and greater than normal or high risk of acquiring a MRSA or SSI, should the person be exposed to MRSA or should they have an invasive surgical procedure. Collectively these 13 SNPs may be thought of as genetic risk factors. Genetic risk factors are in contrast to traditional factors such as exposure and prior disease history. In one embodiment of the invention, the SNPs, or markers are additive and the more markers an individual has, the greater the chance of acquiring a MRSA or an SSI. In one embodiment of the invention the markers are less than additive, and the more markers an individual has does not significantly increase the chance of acquiring a MRSA or an SSI once a single marker is established. In one embodiment of the invention the markers are partially additive, and the more markers an individual has the greater the chance of acquiring a MRSA or SSI, but each additional marker does not add the same weight to the chance of acquiring a MRSA or SSI as an individual with one fewer marker. In one embodiment of the invention the markers are selective, and the more markers an individual has the greater the chance of acquiring a MRSA or SSI infection, but not every marker or SNP adds the same degree of increase in risk, and some markers appear not to be relevant for different types of MRSA.

It is the presence or absence of these SNPs aka genetic markers, aka critical SNP nucleotides, either alone or in combinations, that becomes one of the factors in trying to determine how likely it is that an individual will develop MRSA or SSI, in addition to types of exposure, medical history and sometimes other factors. The presence or absence of these genetic markers in combination with different preventive or treatment options makes these nucleotide bases important for identification of susceptible individuals and the treatment of possibly lethal bacterial infections. It is important to note that the SNPs discussed here may have other purposes. The 13 SNPs listed by number letter and with unique SNP “rs” numbers may or may not play critical roles in other diseases or health issues unrelated to skin and Staph infections, for example, medical conditions associated with Alzheimer's, stomach illness, neurological or pulmonary disease may be completely unrelated to the SNPs identified here even though they also appear to be associated with other conditions. We are not commenting on or claiming the application of these SNPs to unrelated conditions or diseases. The disclosed SNPs are open for other studies, correlations, uses, interpretations and further investigations. We do assert these SNP have application beyond MRSA but may also apply to SSI.

Each polymorphism (SNP) that indicates a susceptibility to MRSA infection and its gene or location is identified and discussed below. The SNP information below provides the “indicator positions” or “critical SNP positions” of SNPs “a1” to “m13” and it explains what nucleotide bases are “controls” or “normal” and what nucleotide bases are indicative of greater risk of acquiring a MRSA infection, for each critical SNP position.

The “a1” SNP.

SNP/rs2249861 or “a1” is within the FAM 129B Gene.

The gene known as FAM 129B encodes a protein that has a predicted molecular mass of 83 kDa, and contains a pleckstrin homology domain and a proline-rich region that contains six serine phosphorylation sites (Chen et al (2011) J. Biol. Chem. 286(12):10201-10209; Old et al. (2009) Mol. Cell 34: 115-131). Phosphorylation has been associated with MAP kinase signaling cascade; in melanoma cells the MAP kinase pathway was active and the FAM129B protein was localized throughout the cytoplasm. When the MAP kinase pathway was inhibited, the FAM129B protein migrated to the cell membrane and melanoma cell migration through a collagen matrix was inhibited. (Old et al., p. 125). Subsequent work found that FAM129B was cytoplasmically localized in actively growing HeLa cells, but appeared to be localized at cell-cell junctions on the plasma membrane when the HeLa cells achieved confluence, and throughout the cell membrane during telophase. (Chen et al. pp. 10203-10204.) FAM129B also inhibited apoptosis in HeLa cells treated with TNFα or CHX, compared with knockdown FAM129B HeLa cells silenced with siRNA sequences specific to FAM129B. A recent investigation of the corresponding Fam129B protein in mice showed that Fam129B is expressed in the epidermal keratinocytes in embryonic and adult mice. Fam129B-knockout mice exhibited delayed wound healing and had altered expression of several wound-repair and cell-motility related genes (Oishi et al. (published online Sep. 11, 2012), J. Biochem. doi:10.1093/jb/mvs100).

In our studies all participants for this allele were homozygous; however we expect to find and have data to support our finding that both homozygous and nonhomozygous individuals for this and other alleles are at greater risk of developing MRSA or SSI if they have even one of two alleles that have a “MRSA indicated risk factor nucleotide”. In the case of the “a1” SNP it has an A or adenine in the high risk SNP position when compared to those who are normal. The full gene sequence of Fam129B with A or adenine at position 17 indicating greater risk of a MRSA or SSI infection is in SEQ ID NO 1. “Normals” or “controls” are those not at greater risk of developing MRSA or SSI, in the case of the “a1” SNP that means they have a C or cytosine in this SNP position. The full gene sequence of Fam129B with C or cytosine at position 17 indicating normal risk of a MRSA or SSI infection also called a control, see SEQ ID NO 2. See sequence below for the Fam129B SNP where both MRSA susceptible and control are described with shorter sequences.

The precise location of the SNP within the Fam129B gene is indicated here and in the sequence listing. The SNP can be identified and located using the sequences or fragments of the sequences provided below or using the information in the sequence listing which identifies and places the SNP within the full gene sequence. SEQ ID NOs 1 and 2 have the full Fam129B sequence. SEQ ID NO 1 shows the MRSA SNP and SEQ ID NO 2 shows the control SNP. Related sequences or probes could be made by one skilled in the art. The complete sequence of the Fam129B gene is in the sequence listing and the polymorphism for the partial gene sequence of SEQ ID NOs 3 and 4 are indicated at position 17 and the nucleotide is defined as being either an A (infection risk more likely) or a C (infection risk normal or control.) See the sequence below for the indicator SNP or critical SNP located within the Fam129B gene. SEQ ID NO 3 shows an A or adenosine (bracketed) for those with an increased risk of MRSA and SEQ ID NO 4 (control) shows a C or cytosine (bracketed) in the SNP position for those not with an increased risk of developing MRSA or SSI. If the reference allele is identified as MRSA then a person who has the high risk nucleotide in this position is at greater than normal risk of developing MRSA or SSI. SNP/rs2249861 or “a1”

| Reference | Affymetrix |
|---|---|
| allele | and GRCh38.p2 Primary Assembly Sequence |
| MRSA | GGGGGCAAGT TAGTCA[A]CCT GTCTGAGTCT TAG |
| | (SEQ ID NO 3) |
| Controls | GGGGGCAAGT TAGTCA[C]CCT GTCTGAGTCT TAG |
| | (SEQ ID NO 4) |

Fam129B sequence above shows the SNP in brackets. Features of Fam129B (SNP/rs2249861) MRSA patients.

Note that SEQ ID NOs 1 and 2 provide the full gene sequence for the Fam129B gene, MRSA susceptible in SEQ ID NO 1 and the control or normal in SEQ ID NO 2. For the remainder of the SNPs we only provide 1 sequence for the full gene sequence for the gene and we describe where in the full gene sequence the SNP can be found and the location of the SNP and what nucleotide is present in normals or controls and what nucleotide is present in the MRSA or SSI susceptible person.

The “b2” SNP.

SNP/rs2352969 or “b2” is within the RBM6 Gene.

This gene, also called “RNA Binding Motif 6,” is a tumor suppressor gene (See SEQ ID NO 5) that can promote cell growth and it may be involved in wound healing. Individuals are usually homozygous for this allele but both homozygous and nonhomozygous individuals are at greater risk of developing MRSA or SSI if they have a C or cytosine in the SNP position when compared to those who are normal. “Normals” or “controls” are those not at greater risk of developing MRSA or SSI, and they have a G or guanine in this SNP position. See sequence and reference sequence below for the RBM6 SNP where both MRSA susceptible and control are described.

The precise location of the SNP within the RBM6 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the RBM6 gene is provided in the sequence listing, with the polymorphism indicated at position 26162 and the nucleotide is defined as being either a C or a G. See the sequence below which shows the region around and the SNP located within the RBM6 gene. SEQ ID NO 6 has a C or cytosine (bracketed) in the SNP position for those with an increased risk of MRSA or SSI and SEQ ID NO 7 (control) shows a G or guanine (bracketed) for those not at increased risk of MRSA. See MRSA sequence and control sequence below for the RBM6 SNP where both MRSA susceptible and control are indicated.

If the reference allele is labeled MRSA then a person who has the bracketed nucleotide in this position is at greater than normal risk of developing MRSA or SSI.

SNP/rs2352969 or “b2”

| Reference | Affymetrix |
|---|---|
| allele | and GRCh38.p2 Primary Assembly Sequence |
| MRSA | TCTTGAACTG AGTGACCCAG AATCA[C]TTGT |
| | CCTTTGAATT TTAGTATAGT A (SEQ ID NO 6) |
| Controls | TCTTGAACTG AGTGACCCAG AATCA[G]TTGTT |
| | CCTTTGAATT TTAGTATAGT A (SEQ ID NO 7) |

RBM6 sequence above, see SNP in brackets. Features of RBM6 (SNP/rs2352969) MRSA patients: 10 of 11 are homozygous for the allele; 1 is heterozygous. Affymetrix and GRCh38.p2 Primary Assembly Sequence.

The “c3” SNP.

SNP/rs12758910 or “c3” within the KANK 4 Gene.

The gene known as KANK 4 is a homologue of genes involved in the regulation of actin polymerization and cell motility. Individuals are usually homozygous for this allele but both homozygous and nonhomozygous individuals are at greater risk of developing MRSA or SSI if they have a G or guanine in the SNP position when compared to those who are normal. “Normals” or “controls” are those not at greater risk of developing MRSA or SSI, if they have a T or thymine in this SNP position. See MRSA sequence and control reference sequence below for the KANK 4 SNP where both MRSA susceptible and control are described.

The precise location of the SNP within the KANK 4 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the KANK 4 gene is in the sequence listing as SEQ ID NO 8 and the polymorphism is indicated at position 76547 and the nucleotide at this position is either a G or a T. See the sequence below for the indicator SNP or critical SNP located within the KANK 4 gene. SEQ ID NO 9 shows a G or guanine (bracketed) in the SNP position for those with an increased risk of developing MRSA or SSI. SEQ ID NO 10 (control) shows a T or thymine (bracketed) for those not at increased risk of MRSA. If the reference allele is labeled MRSA then a person who has the bracketed nucleotide in this position is at greater than normal risk of developing MRSA or SSI.

SNP/rs12758910 or “c3”

| Reference | Affymetrix |
|---|---|
| allele | and GRCh38.p2 Primary Assembly Sequence |
| MRSA | CACCTGCCAA GACAGAGACT GGAGA[G]AAAG |
| | CCTCACTTGC AGAGAACCAGC (SEQ ID NO 9) |
| Controls | CACCTGCCAA GACAGAGACT GGAGA[T]AAAG |
| | CCTCACTTGCA GAGAACCAGC (SEQ ID NO 10) |

KANK 4 sequence above shows the SNP in brackets. Features of KANK 4 (SNP/rs12758910) MRSA patients: 10 of 11 are homozygous for the G allele; 1 is heterozygous. Controls: 2 of 3 are homozygous for the T allele; 1 is heterozygous.

The next group of markers is what we call the “collagen markers” or collagen SNPs. The collagen markers are the following: “d4” known as SNP/rs2642572; “e5” known as SNP/rs3793038; “f6” known as SNP/rs3805987; “g7” known as SNP/rs9454944; “h8” known as SNP/rs978291; and “i9” known as SNP/rs978290 are all within either the Col 13A1 gene (“d4”) or the Col 19A1 gene (“e5, f6, g7, h8 and i9”) both genes code for collagen. These 6 critical SNPs in some respects behave the same but in other respects may behave differently from the other markers we have identified here, but they are still associated with SSI or MRSA infections. They may be thought of as belonging to their own unique group. This group of genes is known for making proteins related to collagens, which are involved with cartilage, bone, tendon and skin.

The “d4” SNP.

SNP/rs2642572 or “d4” Between the C10orf35 Isoform X1 Gene and the Col 13A1 Gene.

The gene known as Col 13A1 is a gene that codes for collagen, a major component of the skin. Individuals are usually homozygous for this allele but both homozygous and nonhomozygous individuals have a greater risk of developing MRSA or SSI if they have a C or cytosine in the SNP position when compared to those who are normal. The full sequence for the C10orf35 isoform X1 gene is provided in SEQ ID NO 11. See sequence and reference sequence below for d4 or SNP/rs2642572 where both MRSA susceptible and control are described.

The precise location of the SNP associated with the C10orf35 isoform X1 gene is indicated below and in the sequence listing of the full gene, SEQ ID NO 11. The polymorphism is indicated at position 159166 of the full gene and the nucleotide at that position is defined as being either a G or an A. The SNP in a patient can be identified and located using the sequences or fragments of the sequences provided in the sequence below, or SNP/rs2642572. SEQ ID NO 11 shows the SNP within the full gene sequence. Related sequences or probes could be made by one skilled in the art. See the sequence below for the indicator SNP, or critical SNP, located outside of the C10orf35 isoform X1 gene. SEQ ID NO 12 shows a G or guanine (bracketed) present in the partial gene sequence at the 26^{th} position for those with an increased risk of developing MRSA and or SSI (MRSA). SEQ ID NO 13 (control) shows an A or adenine (bracketed) is present in the partial gene sequence at the 26^{th} position for those not at increased risk of MRSA. If the reference allele is labeled “MRSA” then a person who has the bracketed nucleotide in this position is at greater than normal risk of developing MRSA or SSI.

SNP/rs2642572 or “d4”

| Reference | |
|---|---|
| allele | GRCh38.p2 Primary Assembly Sequence |
| MRSA | GCTGCAGGAG CCTCCAACAA AGCCA[G]ACTG |
| | TGACAGGGTG GAACCCCAGA G (SEQ ID NO 12) |
| Controls | GCTGCAGGAG CCTCCAACAA AGCCA[A]ACTG |
| | TGACAGGGTG GAACCCCAGA G (SEQ ID NO 13) |

C10orf35 isoform X1 (SNP/rs2642572) sequence above, see SNP in brackets. Features of SNP/rs2642572, it has 155853 bp at 5′ side: uncharacterized protein C10orf35 isoform X1. 12512 bp at 3′ side: collagen alpha-1(XIII) chain isoform 19. Affymetrix annotated SNP as the reverse complement compared to the GRCh38.p2 Primary Assembly; therefore, Affymetrix allele A is GRCh38.p2 Primary Assembly allele T, and Affymetrix allele G is GRCh38.p2 Primary Assembly allele C. MRSA patients: 11 of 11 are homozygous for the C allele Controls: 1 of 3 are homozygous for the T allele; 2 are heterozygous.

The “e5, f6, g7, h8 and i9” SNPs.

The Col 19A1 Gene “e5, f6, g7, h8 and i9”.

The Col 19A1 gene is similar in function to Col 13A1, because it also codes for collagen. However Col 19A1 is different from the other genes reported here because it has 5 SNPs associated with MRSA risk that, either individually or in combination, is associated with an increased risk of MRSA and or SSI. These 5 SNPs are given the following unique reference numbers: SNP/rs3793038 or “e5” (SNP critical position 163341); SNP/rs3805987 or “f6” (SNP critical position 162830); SNP/rs9454944 or “g7” (SNP critical position 162547); SNP/rs978291 or “h8” (SNP critical position 152225); and SNP/rs978290 or “i9” (SNP critical position 151755) and they are described below.

These are what we call “collagen markers” and the so called collagen markers are an example of the “not just MRSA” effect. The markers that are called: “e5” known as SNP/rs3793038, “f6” known as SNP/rs3805987, “g7” known as SNP/rs9454944, “h8” known as SNP/rs978291, and “i9” known as SNP/rs978290 are all within the Col 19A1 gene and these 5 critical SNPs may behave differently from the other markers we have identified. They are thought to belong to their own unique group. This group of genes is known for making proteins related to the family of proteins known as collagens which are involved with cartilage, bone, tendon and skin, certainly other uses than acting as a critical SNP for MRSA and we are not suggesting those other uses are claimed here.

The “e5” SNP.

SNP/rs3793038 or “e5” is within the Col 19A1 Gene.

Individuals are usually homozygous for this allele but both homozygous and nonhomozygous individuals have a greater risk of developing MRSA and or SSI if they have a T or thymine in this SNP in this position when compared to those who are normal. “Normals” or “controls” are those not at greater risk of developing MRSA or SSI, and they have a G or guanine in this SNP position. See MRSA sequence and control reference sequence below for the SNP/rs3793038 SNP where both MRSA susceptible and control are described.

The precise location of SNP/rs3793038 within the Col 19A1 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the Col 19A1 gene is in the sequence listing (SEQ ID NO 14) with the polymorphism indicated at position 163341 and the nucleotide is defined as being either T or G. See the sequence below for the indicator SNP, or critical SNP, (SNP/rs3793038 or e5) located within the Col 19A1 gene. SEQ ID NO 15 shows a T or thymine for those with an increased risk of MRSA or SSI. If the reference allele is labeled “MRSA” then a person who has the bracketed nucleotide in this position is at greater than normal risk of developing MRSA or SSI. SEQ ID NO 16 (control) shows a G or guanine (bracketed) for those not at increased risk of MRSA.

SNP/rs3793038 or “e5”

| Reference | |
|---|---|
| allele | GRCh38.p2 Primary Assembly Sequence |
| MRSA | TAAAAACAAC ATTTATTAAG [T] GCCTACCGTA |
| | TGCTAAATGT (SEQ ID NO 15) |
| Controls | TAAAAACAAC ATTTATTAAG [G] GCCTACCGTA |
| | TGCTAAATGT (SEQ ID NO 16) |

SNP/rs3793038 or e5 sequence above, see SNP in brackets. Features of Col 19A1 SNP/rs3793038 are Affymetrix annotated SNP as the reverse complement compared to the GRCh38.p2 Primary Assembly; therefore, Affymetrix allele A is GRCh38.p2 Primary Assembly allele T, and Affymetrix allele C is GRCh38.p2 Primary Assembly allele G.

MRSA patients: 11 of 11 are homozygous for the T allele
Controls: 1 of 3 is homozygous for the G allele; 2 are heterozygous

The “f6” SNP.

SNP/rs3805987 or “f6” is within the Col 19A1 Gene.

Individuals are usually homozygous for this allele but both homozygous and nonhomozygous individuals have a greater risk of developing MRSA and or SSI if they have a G or guanine in this SNP in this position when compared to those who are normal. “Normals” or “controls” are those not at greater risk of developing MRSA or SSI, and they have an A or adenine in this SNP position. See MRSA sequence and control reference sequence below for the SNP/rs3805987 where both MRSA susceptible and control are described.

The precise location of the SNP/rs3805987 within the Col 19A1 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the Col 19A1 gene is in the sequence listing (SEQ ID NO 14) with this polymorphism indicated at position 162830 and the nucleotide is defined as being either G or A. See the sequence below for the indicator SNP, or critical SNP, (SNP/rs3805987 or f6) located within the Col 19A1 gene. SEQ ID NO 17 shows a G or guanine for those with an increased risk of MRSA or SSI. If the reference allele is labeled “MRSA” then a person who has the bracketed nucleotide in this position is at greater than normal risk of developing MRSA or SSI. SEQ ID NO 18 (control) shows an A or adenine (bracketed) for those not at increased risk of MRSA or SSI.

SNP/rs3805987 or “f6”

| Reference | |
|---|---|
| allele | GRCh38.p2 Primary Assembly Sequence |
| MRSA | AGCITTTGTC TTGCCTTTAG ATAAC [G] GTTT |
| | CTGCCTCCAC ATTACATGTA G (SEQ ID NO 17) |
| Controls | AGCTTTTGTC TTGCCTTTAG ATAAC [A] GTTT |
| | CTGCCTCCAC ATTACATGTA G (SEQ ID NO 18) |

SNP/rs3805987 or f6 sequence is above. See SNP in brackets. Features of Affymetrix annotated SNP as the reverse complement compared to the GRCh38.p2 Primary Assembly; therefore, Affymetrix allele C is GRCh38.p2 Primary Assembly allele G, and Affymetrix allele T is GRCh38.p2 Primary Assembly allele A. MRSA patients: 11 of 11 are homozygous for the G allele. Controls: 1 of 3 is homozygous for the A allele; 2 are heterozygous.

The “g7” SNP.

SNP/rs9454944 or “g7” is within the Col 19A1 gene.

Individuals are usually homozygous for this allele but both homozygous and nonhomozygous individuals have a greater risk of developing MRSA and or SSI if they have a G or guanine in this SNP in this position when compared to those who are normal. “Normals” or “controls” are those not at greater risk of developing MRSA or SSI, and they have an A or adenine in this SNP position. See MRSA sequence and reference MRSA sequence below for the SNP/rs9454944 where both MRSA susceptible and control are described.

The precise location of the SNP/rs9454944 within the Col 19A1 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the Col 19A1 gene is in the sequence listing (SEQ ID NO 14) with the polymorphism indicated at position 162547 and the nucleotide is defined as being either G or A. See the sequence below for the indicator SNP, or critical SNP, (SNP/rs9454944 or g7) located within the Col 19A1 gene. SEQ ID NO 19 shows a G or guanine for those with an increased risk of MRSA or SSI. If the reference allele is labeled “MRSA” then a person who has the bracketed nucleotide in this position is at greater than normal risk of developing MRSA or SSI. SEQ ID NO 20 (control) shows an A or adenine (bracketed) for those not at increased risk of MRSA or SSI.

SNP/rs9454944 or “g7”

| Reference | Affymetrix |
|---|---|
| allele | and GRCh38.p2 Primary Assembly Sequence |
| MRSA | TATACCAATG CAATCTGTAC TAGAG[G]ATAC |
| | AAAAATATTG AAATCACAAA A (SEQ ID NO 19) |
| Controls | TATACCAATG CAATCTGTAC TAGAG[A]ATAC |
| | AAAAATATTG AAATCACAAA A (SEQ ID NO 20) |

Sequence of SNP/rs9454944 or g7 is above. See SNP in brackets. Features of MRSA patients: 11 of 11 are homozygous for the G allele. Controls: 1 of 3 is homozygous for the A allele; 2 are heterozygous.

The “h8” SNP.

SNP/rs978291 or “h8” is within the Col 19A1 Gene.

Individuals are usually homozygous for this allele but both homozygous and nonhomozygous individuals have a greater risk of developing MRSA and or SSI if they have a T or thymine in this SNP in this position when compared to those who are normal. “Normals” or “controls” are those not at greater risk of developing MRSA or SSI, and they have a C or cytosine in this SNP position. See MRSA sequence and control reference sequence below for the SNP/rs978291 where both MRSA susceptible and control are described.

The precise location of the SNP/rs978291 within the Col 19A1 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the Col 19A1 gene is in the sequence listing (SEQ ID NO 14) with the polymorphism indicated at position 152225 and the nucleotide is defined as being either T or C. See the sequence below for the indicator SNP, or critical SNP (SNP/rs978291) located within the Col 19A1 gene. SEQ ID NO 21 shows a T or thymine for those with an increased risk of MRSA or SSI. If the reference allele is labeled “MRSA” then a person who has the bracketed nucleotide in this position is at greater than normal risk of developing MRSA or SSI. SEQ ID NO 22 (control) shows a C or cytosine (bracketed) for those not at increased risk of MRSA or SSI.

SNP/rs978291 or “h8”

| Reference | Affymetrix |
|---|---|
| allele | and GRCh38.p2 Primary Assembly Sequence |
| MRSA | AACTGCAGCT GAGTCCTACT GGAAA[T]CCTC |
| | TGAGGAAGCC TGTAGAATGT G (SEQ ID NO 21) |
| Controls | AACTGCAGCT GAGTCCTACT GGAAA[C]CCTC |
| | TGAGGAAGCC TGTAGAATGT G (SEQ ID NO 22) |

Sequence of SNP/rs978291 or h8 is above. See SNP in brackets. Features of MRSA patients: 11 of 11 are homozygous for the T allele. Controls: 1 of 3 is homozygous for the C allele; 2 are heterozygous.

The “i9” SNP.

SNP/rs978290 or “i9” is within the Col 19A1 Gene.

Individuals are usually homozygous for this allele but both homozygous and nonhomozygous individuals have a greater risk of developing MRSA and or SSI if they have an A or adenine in this SNP in this position when compared to those who are normal. “Normals” or “controls” are those not at greater risk of developing MRSA or SSI, and they have a G or guanine in this SNP position. See MRSA sequence and control reference sequence below for the SNP/rs978290 where both MRSA susceptible and control are described.

The precise location of the SNP/rs978290 within the Col 19A1 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the Col 19A1 gene is in the sequence listing (SEQ ID NO 14) with the polymorphism indicated at position 151755 and the nucleotide is defined as being either A or G. See the sequence below for the indicator SNP, or critical SNP (SNP/rs978290) located within the Col 19A1 gene. SEQ ID NO 23 shows an A or adenine for those with an increased risk of MRSA or SSI. If the reference allele is labeled “MRSA” then a person who has the bracketed nucleotide in this position is at greater than normal risk of developing MRSA or SSI. SEQ ID NO 24 (control) shows a G or guanine (bracketed) for those not at increased risk of MRSA or SSI.

SNP/rs978290 or “19”

| Reference | |
|---|---|
| allele | GRCh38.p2 Primary Assembly Sequence |
| MRSA | TTCTTGGTAG AAAGCAAGGT TTTTG[A]GAGT |
| | AAAAGCAGAA GTCATAATAC C (SEQ ID NO 23) |
| Controls | TTCTTGGTAG AAAGCAAGGT TTTTG[G] GAGT |
| | AAAAGCAGAA GTCATAATAC C (SEQ ID NO 24) |

Sequence of SNP/rs978290 or i9. See SNP in brackets. Features of Affymetrix annotated SNP as the reverse complement compared to the GRCh38.p2 Primary Assembly; therefore, Affymetrix allele C is GRCh38.p2 Primary Assembly allele, G and Affymetrix allele T is GRCh38.p2 Primary Assembly allele A. MRSA patients: 11 of 11 are homozygous for the A allele. Controls: 1 of 3 is homozygous for the G allele; 2 are heterozygous.

The “j10” SNP.

SNP/rs1131891 or “j10” is within the IGSF8 Gene.

This gene, IGFS8, codes for immunoglobulin superfamily protein 8, which is linked to cell motility, polarity, cell adhesion, migration of leukocytes between cells, and infection with parasites. Individuals are usually homozygous for this allele but both homozygous and nonhomozygous individuals have a greater risk of developing MRSA and or SSI if they have a G or guanine SNP in this position when compared to those who are normal. “Normals” or “controls” are those not at greater risk of developing MRSA or SSI, and they have an A or adenine in this SNP position. See MRSA sequence and control reference sequence below for the SNP/rs1131891 where both MRSA susceptible and control are described.

The precise location of the SNP/rs1131891 or j10, located within the IGSF8 gene, is indicated below and in the sequence listing. The complete sequence of the IGSF8 gene is in the sequence listing (SEQ ID NO 25) with the polymorphism indicated at position 6692 and the nucleotide is defined as being either G or A. The SNP can be identified and located using the sequences or fragments of the sequences provided below or in the sequence listing. The location of the SNP within the full and partial gene sequences is indicated. Related sequences or probes could be made by one skilled in the art. See the sequence below for the indicator SNP/rs1131891, or critical SNP “j10” located within the IGSF8. SEQ ID NO 26 shows a G or guanine for those with an increased risk of MRSA or SSI. If the reference allele is labeled “MRSA” then a person who has the bracketed nucleotide in this position is at greater than normal risk of developing MRSA or SSI. SEQ ID NO 27 (control) shows an A or adenine (bracketed) for those not at increased risk of MRSA or SSI.

SNP/rs1131891 or “j10”

| Reference | |
|---|---|
| allele | Sequence |
| MRSA | ACCCTTTTCA GGTCTTGCAG GTGTC[G]ACT |
| | GTCTTCCGGC CCAGCTCCAA GC (SEQ ID NO 26) |
| Controls | ACCCTTTTCA GGTCTTGCAG GTGTC[A]ACT |
| | GTCTTCCGGC CCAGCTCCAA GC (SEQ ID NO 27) |

The “k11” SNP.

SNP/rs127711411 or “k11” is within the ADARB2 Gene.

This gene, ADARB2, known as, adenosine deaminase RNA-specific B2, is linked to neurologic diseases but, oddly, has no known link to the skin or the immune system. Individuals are usually homozygous for this allele but both homozygous and nonhomozygous individuals have a greater risk of developing MRSA and or SSI if they have an A or adenine SNP in this position when compared to those who are normal. “Normals” or “controls” are those not at greater risk of developing MRSA or SSI, and they have a G or guanine in this SNP position. See MRSA sequence and control reference sequence below for the SNP/rs127711411 where both MRSA susceptible and control are described.

The precise location of the SNP/rs127711411 within the ADARB2 gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the ADARB2 gene is in the sequence listing (SEQ ID NO 28) with the polymorphism indicated at position 223202 and the nucleotide is defined as being either A or G. See the sequence below for the indicator SNP/rs127711411, or critical SNP “k11”, located within the ADARB2 gene. SEQ ID NO 29 shows an A or adenine for those with an increased risk of MRSA or SSI. If the reference allele is labeled “MRSA” then a person who has the bracketed nucleotide in this position is at greater than normal risk of developing MRSA or SSI. SEQ ID NO 30 (control) shows a G or guanine (bracketed) for those not at increased risk of MRSA or SSI.

SNP/rs127711411 or “k11”

| Reference | |
|---|---|
| allele | Sequence |
| MRSA | ACTAGATGCC CTGAAAATCA [A] TCTGCAGCCC |
| | ACTCCAGCCT (SEQ ID NO 29) |
| Controls | ACTAGATGCC CTGAAAATCA [G] TCTGCAGCCC |
| | ACTCCAGCCT (SEQ ID NO 30) |

The “l12” SNP.

SNP/rs4773794 or “l12” is within the DCT Gene.

This gene, DCT, known as, dopachrome tautomerase, is found in skin cells and is melanogenic. The DCT-phenotype is associated with enhanced neurotization in benign nevi and with ulceration in thin malignant melanomas. DCT is a mediator of a melanoma stress-resistant pathway, and an antiapoptotic molecule. Individuals are usually homozygous for this allele but both homozygous and nonhomozygous individuals have a greater risk of developing MRSA and/or SSI if they have a C or cytosine in this SNP position when compared to those who are normal. “Normals” or “controls” are those not at greater risk of developing MRSA or SSI, and they have a T or thymine in this SNP position. See MRSA sequence and control reference sequence below for the SNP/rs4773794 where both MRSA susceptible and control are described.

The precise location of the SNP/rs4773794 or l12 within the DCT gene is indicated below and in the sequence listing. The SNP can be identified and located using the sequences or fragments of the sequences provided below or using the information in the sequence listing which identifies and places the SNP within the full gene sequence. Related sequences or probes could be made by one skilled in the art. The complete sequence of the DCT gene is in the sequence listing (SEQ ID NO 31) with the polymorphism indicated at position 81032 and the nucleotide is defined as being either C or T. See the sequence below for the indicator SNP/rs4773794, or critical SNP “l12”, located within the DCT sequence. SEQ ID NO 32 shows a C or cytosine for those with an increased risk of MRSA or SSI. If the reference allele is labeled “MRSA” then a person who has the bracketed nucleotide in this position is at greater than normal risk of developing MRSA or SSI. SEQ ID NO 33 (control) shows a T or thymine (bracketed) for those not at increased risk of MRSA or SSI.

SNP/rs4773794 or “l12”

| Reference | |
|---|---|
| allele | Sequence |
| MRSA | CTGGGAATGT GTCTAGGAAA C[C]CTCTAGTG |
| | CTTAGTCCAC TTG (SEQ ID NO 32) |
| Controls | CTGGGAATGT GTCTAGGAAA C[T]CTCTAGTG |
| | CTTAGTCCAC TTG (SEQ ID NO 33) |

Sequence of SNP/rs4773794 or l12, above.

The “m13” SNP.

SNP/rs4619820 or “m13” is Near but Outside of and Between Two Genes.

The SNP/rs4619820 SNP is located between the gene that codes for CCR2 chemokine (C-C motif) receptor-2, also called CCR2 or DDR2 (provided here in the sequence listing as SEQ ID NO 34) and lactoferrin, also known as lactotransferrin (LTF). LTF, lactoferrin or lactotransferrin, stimulates the proliferation and migration of fibroblasts and keratinocytes and enhances the synthesis of extracellular matrix components, such as collagen and hyaluronan. The protein demonstrates a broad spectrum of properties, including regulation of iron homeostasis, host defense against a broad range of microbial infections, anti-inflammatory activity, regulation of cellular growth and differentiation and protection against cancer development and metastasis. Antimicrobial, antiviral, antifungal and antiparasitic activity has been found for this protein and its peptides. CCR2 (sometimes also called DDR2) is a gene that encodes two isoforms of a receptor for monocyte chemoattractant protein-1, a chemokine which specifically mediates monocyte chemotaxis. Monocyte chemoattractant protein-1 is involved in monocyte infiltration in inflammatory diseases such as rheumatoid arthritis as well as in the inflammatory response against tumors. The receptors encoded by this gene mediate agonist-dependent calcium mobilization and inhibition of adenylyl cyclase. This gene is located in the chemokine receptor gene cluster region. Two alternatively spliced transcript variants are expressed by the gene.

Individuals are usually homozygous for this allele but both homozygous and nonhomozygous individuals have a greater risk of developing MRSA and or SSI if they have a G or guanine in this position when compared to those who are normal. “Normals” or “controls” are those not at greater risk of developing MRSA or SSI, and they have A or adenine in this SNP position. See MRSA sequence and control reference sequence below for the SNP/rs4619820 where both MRSA susceptible and control are described. SEQ ID NO 35 is the gene sequence which starts at position 46,360,941 (at the end of the CCR2 gene) and ends at position 46,425,932; the SNP/rs4619820 is contained between these positions, but before the LTF gene, The LTF gene is on Chr3: 46,436,005-46,465,142. The polymorphism is indicated at position 64492 and the nucleotide is defined as being either G or A. See the sequence below for the indicator SNP/rs4619820, or critical SNP “m13” position, located within the sequence. SEQ ID NO 36 shows a G or guanine for those with an increased risk of MRSA or SSI. If the reference allele is labeled “MRSA” then a person who has the bracketed nucleotide in this position is at greater than normal risk of developing MRSA or SSI. SEQ ID NO 37 (control) shows a A or adenine (bracketed) for those not at increased risk of MRSA or SSI.

SNP/rs4619820 or “m13”

Reference

| allele | Sequence |
|---|---|
| MRSA | GATTACAGGC ATGAGACACC GCATG[G]CCTG |
| | CACCTATAGT CCCAGCTATC A (SEQ ID NO 36) |
| Controls | GATTACAGGC ATGAGACACC GCATG[A]CCTG |
| | CACCTATAGT CCCAGCTATC A (SEQ ID NO 37) |

Sequence for SNP/rs4619820 or m13, is located between two genes. Features: The location of this SNP is described as located at the following number of base pairs between the two genes. This SNP is 15818 bp at 5′ side of DDR2 or C-C chemokine receptor-like 2 isoform 1 and 10263 bp at 3′ side: lactotransferrin isoform 1 precursor or LTF.

SEQ ID NOs 36 and 37 span 25 nucleotide sequences before and after the SNP.

Combinations of SNPs, Critical SNP Locations and Critical SNP Nucleotides

The SNPs presented above are given a letter/number abbreviation for use and reference above and in the list below, a1-m13, are 13 useful SNPs discussed and claimed in unique combinations and procedures herein.

a1) is SNP/rs2249861 within the FAM 129B gene;
b2) is SNP/rs2352969 within the RBM6 gene;
c3) is SNP/rs12758910 within the KANK 4 gene;
d4) is SNP/rs2642572 outside of and between two genes, C10orf35 isoform X1 and Col 13A1;
e5) is SNP/rs3793038 within the Col 19A1 gene;
f6) is SNP/rs3805987 within the Col 19A1 gene;
g7) is SNP/rs9454944 within the Col 19A1 gene;
h8) is SNP/rs978291 within the Col 19A1 gene;
i9) is SNP/rs978290 within the Col 19A1 gene;
j10) is SNP/rs1131891 within the IGSF8 gene;
k11) is SNP/rs127711411 within the ADARB2 gene;
l12) is SNP/rs4773794 is within the DCT gene; and
m13) is SNP/rs4619820 outside of and between two genes, CCR2 and LTF.

In one embodiment of the invention each SNP is considered, evaluated and used individually and separately. In another embodiment of the invention two SNPs are considered, evaluated and used in combinations of two or more. Using the letter number codes above the following combinations are enumerated:

Several different embodiments, examples and applications of the invention are provided below. In the pages immediately below we provide many examples of actual combinations of SNPs using the abbreviations above, e.g. SNP/rs2249861 within the FAM 129B gene is labeled “a1” and SNP/rs2352969 within the RBM6 gene labeled “b2”. Here we also describe every single combination for 2 SNPs and 3 SNPs to clearly show the progression and to show how to determine every combination for 2, 3, 4, 5, 6, 7, 8, 9, 10, 11, 12 and 13 combinations. There is only one way to make all thirteen combinations of course and that is to include all 13 SNPs. We also note that because of the heterozygous nature of many of the genes, many of the SNPs may appear as duplicates in any diagnostics or treatments.

The general equation used to determine the number of combination is C(n,r)=n!/(r!(n−r)!); where n is the number of possible combinations, here 13 and r is the number of SNPs in the combination, i.e., the number of elements. It is otherwise known as the “n choose r” or “n choose k” equation. Here there are 13 different SNPs that could be used either alone or in different combinations using combinations of 2, 3, 4, 5, 6, 7, 8, 9, 10, 11, 12 and even all 13. Because the number of variables or SNPs is 13 we can say n=13 in the equation. The number of combinations “C” goes from 2-13 and this does not include individual single SNPs which could also be used to identify a person at greater than normal risk of developing a MRSA infection if the person is exposed to MRSA.

Using this formula we calculate the number of possible combinations for 1-13 elements or SNPs as follows:

1=13 (as single SNPs)

2=78

3=286

4=715

5=1287

6=1716

7=1716

8=1287

9=715

10=286

11=78

12=13

13=1 (one of each possible SNP—the same as single SNPs)

Another way to describe these combinations is to use “Markush” type of language commonly used to make and claim generic descriptions of organic compounds where individual parts or elements in the molecules may vary. Here there can be variations in which SNPs are used, including each SNP individually, all 13 SNPs and any and all of the possible combinations of those 13 SNPs. The level of confidence in any diagnostic test developed using these SNPs will likely increase when using more SNPs. We advise the best results would result from using 2 or more SNPS. In order to simplify the discussion we can write the “SNP Markush” as follows:

Combinations of 2 are I(a1, b2, c3, d4, e5, f6, g7, h8, i9, j10, k11, l12, or m13)+I(a1, b2, c3, d4, e5, f6, g7, h8, i9, j10, k11, l12, or m13), where I is a roman numeral that indicates the number of SNP combinations, with duplicates removed. Instead of listing a1, b2, c3, d4, e5, f6, g7, h8, i9, j10, k11, l12, or m13 we could write (a1−m13). So the above equation becomes I(a1−m13)+I(a1−m13) less duplicates=combinations of 2 or 78 possible combinations. We could further use the roman numeral to indicate the number of combinations in which case the above equation becomes “II(a1−m13) less duplicates”=the number of 2 SNP combinations=C(n,r)=n!/(r!(n−r)!); where n is the number of possible combinations, here 2 SNPs would allow for 78 combinations.

In a similar manner we write three (3) SNP combinations either as I(a1−m13)+I(a1−m13)+I(a1−m13) less duplicates as or “III(a1−m3) less duplicates and that would allow for 286 different combinations.”

Using the more abbreviated expression, four (4) SNP combinations are “IV(a1−m13) less duplicates.” 5-13 combinations would be: V(a1−m13) less duplicates or 1287 combinations, VI(a1−m13) less duplicates or 1716 combination, VII(a1−m3) less duplicates or 1716 combination, VIII(a1−m3) less duplicates or 1287 combinations, IX(a1−m13) less duplicates or 715 combinations, X(a1−m13) less duplicates or 286 combinations, XI(a1−m13) less duplicates or 78 combinations, X12(a1−m13) less duplicates or 13 combinations.

In the two sections below we have written out and expanded the Markush formulations given above to show each individual combination. This exercise is a simple one that begins with the lowest alphabet/number combination for each number of combinations desired and then replaces one alpha/number at a time until all are shown. Thus two (2) combinations begin with a1+b2, three (3) combinations begin with a1+b2+c3, four (4) combinations begin with a1+b2+c3+d4 etc. All of the possibilities for two (2) combinations and three (3) combinations are provided below. With this guidance, and the formula provided one skilled in the art should have the written description and enablement needed to know and understand each and every combination given the thirteen (13) SNPs.

Two (2) combinations are as follows. All combination of two SNPS are enumerated and described in abbreviated form as follows:

a1+b2, a1+c3, a1+d4, a1+e5, a1+f6, a1+g7, a1+h8, a1+i9, a1+j10, a1+k11; a1+l12; a1+m13;

b2+c3, b2+d4, b2+e5, b2+f6, b2+g7, b2+h8, b2+i9, b2+j10, b2+k11, b2+l12, b2+m13;
c3+d4, c3+e5, c3+f6, c3+g7, c3+h8, c3+i9, c3+j10, c3+k11, c3+l12, c3+m13;
d4+e5, d4+f6, d4+g7, d4+h8, d4+i9; d4+j10; d4+k11, d4+l12, d4+m13;
e5+f6, e5+g7, e5+h8, e5+i9, e5+j10; e5+k11; e5+l12, e5+m13;
f6+g7, f6+h8, f6+i9; f6+j10; f6+k11, f6+l12, f6+m13;
g7+h8, g7+i9, g7+j10, g7+k11, g7+l12, g7+m13;
h8+i9, h8+j10, h8+k11, h8+l12, h8+m13;
i9+j10, i9+k11, i9+l12, 19+m13;
j10+k11, j10+l12, j10+m13;
k11+l12, k11+m13.

Three (3) combinations are as follows. All combination of three SNPS are enumerated and described in abbreviated form as follows:

a1+b2+c3, a1+b2+d4, a1+b2+e5, a1+b2+f6, a1+b2+g7, a1+b2+h8, a1+b2+i9, a1+b2+j10, a1+b2+k11, a1+b2+l12, a1+b2+m13;

a1+c3+d4, a1+c3+e5, a1+c3+f6, a1+c3+g7, a1+c3+h8, a1+c3+i9, a1+c3+j10, a1+c3+k11, a1+c3+l12, a1+c3+m13;
a1+d4+e5, a1+d4+f6, a1+d4+g7, a1+d4+h8, a1+d4+i9, a1+d4+j10, a1+d4+k11, a1+d4+l12, a1+d4+m13;
a1+e5+f6, a1+e5+g7, a1+e5+h8, a1+e5+i9, a1+e5+j10, a1+e5+k11, a1+e5+l12, a1+e5+m13;
a1+f6+g7, a1+f6+h8, a1+f6+i9, a1+f6+j10, a1+f6+k11, a1+f6+l12, a1+f6+m13;
a1+g7+h8, a1+g7+i9, a1+g7+j10, a1+g7+k11, a1+g7+l12, a1+g7+m13;
a1+h8+i9, a1+h8+j10, a1+h8+k11, a1+h8+l12, a1+h8+m13;
a1+i9+j10, a1+i9+k11, a1+i9+l12, a1+i9+m13;
a1+j10+k11, a1+j10+l12, a1+j10+m13;
a1+k11+l12, a1+k11+m13;
a1+l12+m13,

b2+c3+d4, b2+c3+e5, b2+c3+f6, b2+c3+g7, b2+c3+h8, b2+c3+i9, b2+c3+j10, b2+c3+k11, b2+c3+l12, b2+c3+m13;

b2+d4+e5, b2+d4+f6, b2+d4+g7, b2+d4+h8, b2+d4+i9, b2+d4+j10, b2+d4+k11, b2+d4+l12, b2+d4+m13;
b2+e5+f6, b2+e5+g7, b2+e5+h8, b2+e5+i9, b2+e5+j10, b2+e5+k11, b2+e5+l12, b2+e5+m13;
b2+f6+g7, b2+f6+h8, b2+f6+i9, b2+f6+j10, b2+f6+k11, b2+f6+l12, b2+f6+m13;
b2+g7+h8, b2+g7+i9, b2+g7+j10, b2+g7+k11, b2+g7+l12, b2+g7+m13;
b2+h8+i9, b2+h8+j10, b2+h8+k11, b2+h8+l12, b2+h8+m13;
b2+i9+j10, b2+i9+k11, b2+i9+l12, b2+i9+m13;
b2+j10+k11, b2+j10+l12, b2+j10+m13;
b2+k11+l12, b2+k11+m13;
b2+l12+m13;

c3+d4+e5, c3+d4+f6, c3+d4+g7, c3+d4+h8, c3+d4+i9, c3+d4+j10, c3+d4+k11, c3+d4+l12, c3+d4+m13;

c3+e5+f6, c3+e5+g7, c3+e5+h8, c3+e5+i9, c3+e5+j10, c3+e5+k11, c3+e5+l12, c3+e5+m13;
c3+f6+g7, c3+f6+h8, c3+f6+i9, c3+f6+j10, c3+f6+k11, c3+f6+l12, c3+f6+m13;
c3+g7+h8, c3+g7+i9, c3+g7+j10, c3+g7+k11, c3+g7+l12, c3+g7+m13;
c3+h8+i9, c3+h8+j10, c3+h8+k11, c3+h8+l12, c3+h8+m13;
c3+i9+j10, c3+i9+k11, c3+i9+l12, c3+i9+m13;
c3+j10+k11, c3+j10+l12, c3+j10+m13;
c3+k11+l12, c3+k12+m13;
c3+k12+m13;

d4+e5+f6, d4+e5+g7, d4+e5+h8, d4+e5+i9, d4+e5+j10, d4+e5+k11, d4+e5+l12, d4+e5+m13;

d4+f6+g7, d4+f6+h8, d4+f6+i9, d4+f6+j10, d4+f6+k11, d4+f6+l12, d4+f6+m13;
d4+g7+h8, d4+h8+i9, d4+h8+j10, d4+h8+k11, d4+h8+l12, d4+h8+m13;
d4+h8+i9, d4+h8+j10, d4+h8+k11, d4+h8+l12, d4+h8+m13;
d4+i9+j10, d4+i9+k11, d4+i9+l12, d4+i9+m13;
d4+j10+k11, d4+j10+l12, d4+j10+m13;
d4+k11+l12, d4+k11+m13;
d4+l12+m13;

e5+f6+g7, e5+f6+h8, e5+f6+i9, e5+f6+j10, e5+f6+k11, e5+f6+l12, e5+f6+m13;

e5+g7+h8, e5+g7+i9, e5+g7+j10, e5+g7+k11, e5+g7+l12, e5+g7+m13;
e5+h8+i9, e5+h8+j10, e5+h8+k11, e5+h8+l12, e5+h8+m13;
e5+i9+j10, e5+i9+k11, e5+i9+l12, e5+ki9+m13;
e5+j10+k11, e5+j10+l12, e5+j10+m13;
e5+k11+l12, e5+k11+m13;
e5+l12+m13;

f6+g7+h8, f6+g7+i9, f6+g7+j10, f6+g7+k11, f6+g7+l12, f6+g7+m13;

f6+h8+i9, f6+h8+j10, f6+h8+k11, f6+h8+l12, f6+h8+m13;
f6+i9+j10, f6+i9+k11, f6+i9+l12, f6+g7+m13;
f6+j10+k11, f6+j10+l12, f6+j10+m13;
f6+k11+l12, f6+k11+m13;
f6+l12++m13;

g7+h8+i9, g7+h8+j10, g7+h8+k11, g7+h8+l12, g7+h8+m13;

g7+i9+j10, g7+i9+k11, g7+i9+l12, g7+i9+m13;
g7+j10+k11, g7+j10+l12, g7+j10+m13;
g7+k11+l12, g7+k11+m13;
g7+l12+m13;

h8+i9+j10, h8+i9+k11, h8+i9+l12, h8+i9+m13;

h8+j10+k11, h8+j10+l12, h8+j10+m13;
h8+k11+l12, h8+k11+m13;
h8+l12+m13;

i9+j10+k11, i9+j10+l12, i9+j10+m13;

i9+k11+l12, i9+k11+m13;
i9+l12+m13;

j10+k11+l12, j10+k11+m13;

j10+l12+m13;

k11+l12+m13.

All single SNPs and combinations of 2, 3, 4, 5, 6, 7, 8, 9, 10, 11, 12 and 13 SNPs combined in any number and any combination together are described and claimed. Thus, a1 can be used in any combination with any of the following in combinations of 2-12 including: b2; c3; d4; e5; f6; g7; h8; i9; j10; k11; l12; and m13. Similarly, b2 can be used in any combination with any of the following in combinations of 2-12 including: a1; c3; d4; e5; f6; g7; h8; i9; j10; k11; l12; and m13. Similarly, “c3” can be used in any combination with any of the following in combinations of 2-12 comprising a1; b2; d4; e5; f6; g7; h8; i9; j10; k11; l12; and m13. Similarly, “d4” can be used in any combination with any of the following in combinations of 2-12 including a1; b2; c3; e5; f6; g7; h8; i9; j10; k11; l12; and m13. Similarly, “e5” can be used in any combination with any of the following in combinations of 2-12 including a1; b2; c3; d4; f6; g7; h8; i9; j10; k11; l12; and m13. Similarly, “f6” can be used in any combination with any of the following in combinations of 2-12 including a1; b2; c3; d4; e5; g7; h8; i9; j10; k11; l12; and m13. Similarly, “g7” can be used in any combination with any of the following in combinations of 2-12 including a1; b2; c3; d4; e5; f6; h8; i9; j10; k11; l12; and m13. Similarly “h8” can be used in any combination with any of the following in combinations of 2-12 including a1; b2; c3; d4; e5; f6; g7; i9; j10; k11; l12; and m13. Similarly, “i9” can be used in any combination with any of the following in combinations of 2-12 including a1; b2; c3; d4; e5; f6; g7; h8; j10; k11; l12; and m13. Similarly, “j10” can be used in any combination with any of the following in combinations of 2-12 including a1; b2; c3; d4; e5; f6; g7; h8; i9; k11; l12; and m13. Similarly, “k11” can be used in any combination with any of the following in combinations of 2-12 including a1; b2; c3; d4; e5; f6; g7; h8; i9; j10; 112; and m13. Similarly, “l12” can be used in any combination with any of the following in combinations of 2-12 including a1; b2; c3; d4; e5; f6; g7; h8; i9; j10; k11; and m13. Similarly, “m13” can be used in any combination with any of the following in combinations of 2-12 including a1; b2; c3; d4; e5; f6; g7; h8; i9; j10; k11; and l12.

### Embodiments of the Invention

To practice the methods described herein one begins by obtaining a biological sample from the subject, wherein said biological sample includes at least one oligonucleotide occupying a locus corresponding to any of the SNPs “a1” to “m13” the descriptions and positions are described above; detecting the identity of at least one oligonucleotide occupying a locus corresponding to any of the SNPs a1 to m13, or critical SNPs; determining whether at least one oligonucleotide occupying the locus corresponding to any of the SNPs a1 to m13 is a critical SNP, either control critical SNP or MRSA critical SNP, or indicator SNPs and predicting and assessing the level of risk of the subject developing a MRSA infection depending on whether and how many SNPs the individual has and whether or not they indicate a high risk and how many indicate normal or control risk. If the SNP has the nucleotide type of “normal” or “control” then that indicates that the subject has a normal or lower risk of developing a MRSA infection if exposed to MRSA bacteria than does a MRSA risk or high risk individual when exposed to MRSA bacteria.

We describe embodiments wherein the detecting step further comprises hybridizing at least one oligonucleotide (in the biological sample) occupying a locus corresponding to the SNPs a1 to m13, to an oligonucleotide probe comprising a sequence that is complementary or identical to the sequences of the various sequences in the sequence listing, or to related sequences that would be known to one skilled in the art, under stringency conditions (e.g., high stringency) that can detect the presence of different alleles for the positions of the SNPs a1 to m13.

We describe embodiments wherein the detecting step further comprises evaluating the hybridization of at least one oligonucleotide from the biological sample which corresponds to one of the positions of the SNPs which are described herein. The detecting step further may comprise sequencing the oligonucleotide from the biological sample.

In other embodiments the detecting step further comprises amplifying the oligonucleotide from the biological sample. In some embodiments the amplifying step uses at least one oligonucleotide primer and at least one oligonucleotide from the biological sample occupying a locus corresponding to the nucleotide bases at positions a1 to m13 of the SNPs described here. The oligonucleotide primer may be comprised of DNA.

We describe methods to detect, identify and treat MRSA in all it various stages and forms including the treatments that may be made after it is determined whether the patient has a high or normal risk of developing a MRSA, CA-MRSA infection or HA-MRSA following exposure. One it is determined that the patient has a high risk of developing a MRSA infection, the patient is given anti MRSA antibiotics, and may also be given decolonization, aggressive antibiotic, or recolonization treatments. Once it is determined that the patient has a high risk of developing a MRSA infection, the patient may be given more than one course of anti MRSA antibiotics, decolonization treatments and may be put on high infection alert for any future surgeries. The patient may also be put in isolation or reduced contact zones in order to further decrease the patient's potential exposure to MRSA.

In other embodiments after it is determined that the patient has a normal risk of developing a MRSA infection, the patient is not given any antibiotics and is only treated with incision and drainage, sometimes such a normal risk patient is treated with incision and drainage and given a routine antibiotic treatment that does not include anti MRSA antibiotics.

This application also describes and claims one or more primers or probes to be used to amplify or detect at least one or more nucleotides from a biological sample, wherein one or more nucleotides occupy a locus corresponding to positions indicated in reference to SNPS a1-m13. There may be one or more primers that span the nucleotide positions about the various SNP positions. The primers or probes may be from about 5 to 1500 bases long. Optimally the primers may be from about 5 to about 50 nucleotides in length, or from about 8 or 10 to about 44 nucleotides in length. Many other probe lengths are described herein. Described are probes wherein said one or more probes span the nucleotide positions about the position of SNPs a1-m13. Described are probes having a different disruption energy for one allele as compared to another allele; two probes, wherein the first probe is a sensor probe and the second probe is an anchor probe; and a SNP-specific probe, in addition to methods to make and use these and other primers and probes.

It is important to appreciate that the probes described here are not merely gene fragments, instead they are modified artificial constructs with artificial chemically synthesized indicator regions, or critical SNP locations or locus that are often made radioactive or fluorescent. The source of the genetic material is not relevant to the effectiveness of the probe. The probes are inspired by genetic fragments of genetic material where the fragments identified here were created by artificial chemical reactions but the probes do not exist in nature or in humans.

In some embodiments one or more primers or probes are placed into a kit designed for use by a caregiver who seeks to predict or assess the level of risk of a subject developing a MRSA infection. The kit provides a collection of needed materials that can be easily transported. The kit and its instructions comprise obtaining a biological sample from the subject, wherein said biological sample includes at least one oligonucleotide occupying a locus corresponding to the position of SNPs a1-m13; detecting the identity of said at least one oligonucleotide occupying the locus corresponding to the position of SNPs a1-m13; determining whether said at least one oligonucleotide occupying the locus corresponding to the position of SNPs a1-m13 is a control or a high MRSA risk nucleotide and predicting and assessing the level of risk of the subject developing a MRSA infection.

We disclose methods to monitor and prepare a patient for hospitalization and or surgery, wherein said monitoring and preparing comprise obtaining a biological sample from the patient, wherein said biological sample includes at least one oligonucleotide occupying a locus corresponding to the position of SNPs a1-m13; detecting the identity of the at least one oligonucleotide occupying the locus corresponding to the position of SNPs a1-m13; determining whether said at least one oligonucleotide occupying the locus corresponding to the position of SNPs a1-m13 is a control or a high MRSA risk SNP; predicting and assessing the level of risk of the patient developing a MRSA infection, wherein a “high MRSA risk” SNP indicates that the patient has a high risk of developing a MRSA or CA-MRSA infection and a “Control” SNP occupying the locus corresponding to the position of SNPs a1-m13 indicates that the patient has a normal or average risk of developing a MRSA or CA-MRSA infection; and wherein when the predication and assessment indicates that the patient is at high risk for a MRSA infection, taking appropriate steps and care as one normally skilled in the art would take when operating on a person at high risk of developing a MRSA infection.

One aspect of the inventive method of determining whether a subject is at increased risk of developing MRSA and/or CA-MRSA, or a recurrence of MRSA or CA-MRSA, may include: obtaining a biological sample from the subject, wherein said biological sample contains at least one oligonucleotide comprising a loci corresponding to the position of SNPs a1-m13; detecting the identity of one, all, or any combination of each nucleotide that occurs at the position of the SNPs a1-m13 in said oligonucleotide; and comparing the identity of such nucleotide that occurs at a loci corresponding to SNPs a1-m13 in at least one SNP position to determine whether it or they are a “control” or a high MRSA risk oligonucleotide, wherein the subject is at increased risk of developing MRSA and/or CA-MRSA, or a recurrence of MRSA or CA-MRSA if one or more nucleotides at the loci corresponding to the position of SNPs a1-m13 are the same as the identity of the MRSA high risk or SSI nucleotide at the position of SNPs a1-m13.

Another aspect of the inventive method of determining whether a subject is at increased risk of developing MRSA and/or CA-MRSA, or a recurrence of MRSA or CA-MRSA, is the following method: obtaining a biological sample from the subject, wherein said biological sample contains at least one oligonucleotide comprising a loci corresponding to the position of SNPs a1-m13; detecting the identity of each nucleotide that occurs at a loci corresponding to the position of SNPs a1-m13; and comparing said identity of each nucleotide to the identity of the control and high risk nucleotide at the position of the SNPs a1-m13.

In other aspects, the detecting step may further comprise hybridization of said at least one oligonucleotide comprising a loci corresponding to the position of SNPs a1-m13 to a probe comprising an oligonucleotide comprising a sequence complementary or identical to the nucleotide in the position of SNPs a1-m13, for example, a nucleotide indicating high risk of developing a MRSA infection, under stringency conditions than can detect the presence of different alleles at position 17 of SEQ ID 3 or 4. In other aspects, the detecting step may further comprise hybridization of said at least one oligonucleotide comprising a loci corresponding to the MRSA high risk position of SNPs a1-m13 to a probe comprising an oligonucleotide comprising a sequence complementary or identical to the sequences having either the “MRSA high risk of infection” position of SNPs a1-m13, or normal risk of infection position of SNPs a1-m13, under stringency conditions than can detect the presence of different alleles at the position of SNPs a1-m13. In other aspects, the detecting step further comprises evaluating the hybridization of an oligonucleotide containing a locus corresponding to the position of SNPs a1-m13, for either control or normal SNP positions as we describe in other places in the this description. The SNP position nucleotides derived from said subjects will adhere or align to a probe comprising a sequence complementary or identical to the position of SNPs a1-m13, either MRSA or normal, under stringency conditions, and the use of the probe that can determine and establish the presence of different alleles at the 13 different position of SNPs a1-m13 of said oligonucleotides.

In other aspects, the detecting step may further comprise amplifying at least one oligonucleotide from said biological sample containing a locus corresponding to the position of SNPs a1-m13, wherein said amplifying step uses at least one oligonucleotide primer. In some aspects, the oligonucleotide primer comprises DNA.

In other aspects, the determining step may further comprise using a probe to detect the presence of a locus corresponding to the control position of SNPs a1-m13 in the biological sample. In other aspects, the determining step may further comprise using a probe to detect the presence of a locus corresponding to the high MRSA risk position of SNPs a1-m13 in the biological sample.

In other aspects, the probe may be labeled with a detection signal. In other aspects, the probe may comprise an oligonucleotide having a sequence that is complementary or identical to a region flanking the locus corresponding to the position of SNPs a1-m13.

In other aspects, the method may further comprise treating or providing preventative treatment to the subject with an antibiotic effective against MRSA, when the subject is found to be at increased risk of developing MRSA, CA-MRSA or having a recurrence of MRSA or CA-MRSA, even when the subject is showing no symptoms of MRSA. In other aspects, the method may further comprise treating the subject to remove or prevent colonization by skin-surface or intranasal populations of MRSA, when the subject is found to be at increased risk of developing MRSA, CA-MRSA or having a recurrence of MRSA or CA-MRSA.

In other aspects, the method may further comprise not treating or pre-treating a subject with an antibiotic effective against MRSA, when the subject is found not to be at increased risk of developing MRSA, CA-MRSA or having a recurrence of MRSA or CA-MRSA, even when the results of a survey about increased MRSA or SSI suggest the subject is at high risk of developing MRSA or SSI. In other aspects, the method may further comprise withholding treatment of the subject to remove or prevent colonization by skin-surface or intranasal populations of MRSA, when the subject is found not to be at increased risk of developing MRSA, CA-MRSA or having a recurrence of MRSA or CA-MRSA.

Embodiments of the present invention may comprise a kit for determining whether a subject is at increased risk of developing MRSA, CA-MRSA, or a recurrence of MRSA and/or CA-MRSA, comprising at least one primer for amplification of one or more nucleotides that occur at a loci corresponding to the position of SNPs a1-m13 or a combination thereof from a biological sample from the subject.

Other embodiments of the present invention may comprise a kit for determining whether a subject is at increased risk of developing MRSA, CA-MRSA, or a recurrence of MRSA and/or CA-MRSA, comprising at least one probe for detection of one or more nucleotides that occur at a loci corresponding to the position of SNPs a1-m13 or a combination thereof from a biological sample from the subject.

Single Nucleotide Polymorphisms

Analysis of single nucleotide polymorphisms (SNPs) can be effective in discovering genomic differences (i.e., genotypes) in individuals or populations exhibiting different phenotypes, such as susceptibility or increased risk of contracting certain diseases or syndromes. With the advent of rapid sequencing, amplification and high throughput screening of oligonucleotides, analysis of SNPs can be used to probe individual and population genomes for one or more SNPs that correlate with (are “markers” for) the presence of a certain phenotype. Discovery of one or more reliably correlating markers (among other uses) allows for early diagnosis of potential susceptibility or risk for certain diseases, even where a phenotype is not yet being exhibited in a particular individual, e.g., a late-onset cancer or susceptibility to a disease organism prior to exposure. Here, we disclose a number of SNPs and their locations, either within or associated with certain genes, we disclose what those associated genes are, their full sequence and we provide the sequence of nucleotides immediately upstream and downstream in the genome of all of the SNPs, in order that presentation, identification and comparison of the location of particular SNPs in the genome can be made. We claim and describe how to use the existence of the SNPs to identify the susceptibility, or the risk an individual may face, which can be especially useful when a person is placed in situations where there is traditionally a high risk of acquiring an MRSA or SSI infection.

Often, SNP detection methods can distinguish between homozygous and heterozygous individuals. The presence of zero, zero or one, one, one or two, or two copies of a particular base substitution allele may correlate with a particular phenotype. “Dominant,” “recessive” and “intermediate dominance”/“incomplete dominance” of a particular allele at a locus can be defined as the relative contribution of each allele to the phenotype of a heterozygous individual. For example, where a heterozygous individual carrying one copy of allele A and one copy of allele A′ has the same phenotype as a homozygous AA individual, and a different phenotype from a homozygous A′A′ individual, allele A is dominant over allele A′. Where a heterozygous individual AA′ exhibits a different or intermediate phenotype between the homozygote phenotypes, allele A′ and A are said to exhibit an intermediate or incomplete dominance.

While SNPs occur at particular locations in the genome, presentation, identification and comparison of the location of particular SNPs is aided by inclusion of the sequence of nucleotides immediately upstream and downstream in the genome. SNP detection methods using oligonucleotide hybridization methods may use the same sequence as presented herein as all or part of a primer or probe sequence, and thus such sequences may serve as examples of an appropriate primer or probes for these methods. Persons of skill in the art realize that design of appropriate primers and probes, examples of which are provided below, are not necessarily limited to the sequences listed herein for purposes of presentation, and may be longer or shorter, and include more or less of the upstream and downstream flanking sequence(s), as long as they encompass the location of a SNP.

The oligonucleotide hybridization method typically uses probes of 5, 10, 20, 30, 40, 50, 60, 70, 80, 90 or 100 to 200, 300, 400, 500, 600, 700, 800, 900, 1000, 1100, 1200, 1300, 1400, or 1500 bases long (10 to 100, 20 to 900, 30 to 800, 40 to 700, 50 to 600, 20 to 500, 30 to 400, 40 to 300, 50 to 200, 40 to 100, 40 to 80, 40 to 60, 50 to 100, 50 to 80, bases) or other combinations with more or less bases which have TOM radioactive molecules, fluorescent molecules or some other labeling mechanism. Commonly used markers are ³²P (a radioactive isotope of phosphorus incorporated into the phosphodiester bond in the probe DNA) or Digoxigenin, which is a non-radioactive, antibody-based marker. DNA sequences or RNA transcripts that have moderate to high sequence similarity to the probe are then detected by visualizing the hybridized probe via autoradiography or other imaging techniques. Normally, either X-ray pictures are taken of the filter, or the filter is placed under UV light. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in DNA microarrays refer to DNA covalently attached to an inert surface, such as coated glass slides or gene chips, to which a mobile cDNA target is hybridized.

The probe may be synthesized using a variety of methods, including but not limited to the following: the phosphoramidite method and or it can be generated and labeled by PCR amplification or cloning (both are older methods). In order to increase the in vivo stability of the probe RNA is not used, instead RNA analogues may be used, in particular morpholino-derivatives. Molecular DNA- or RNA-based probes are now routinely used in screening gene libraries, detecting nucleotide sequences with blotting methods, and in other gene technologies, such as nucleic acid and tissue microarrays.

Allelle-specific Oligonucleotide or ASO analysis is only one of the methods used to detect genetic polymorphisms. Direct DNA sequencing can be used to initially characterize the mutation, but is too laborious for routine screening. An earlier method, Restriction Fragment Length Polymorphism (RFLP) didn't need to know the sequence change beforehand, but required that the mutation affect the cleavage site of a Restriction Enzyme. The RFLP assay was briefly adapted to the use of oligonucleotide probes, but this technique was quickly supplanted by ASO analysis of polymerase chain reaction (PCR) amplified DNA. The PCR technique itself has been adapted to detect polymorphisms, as allele-specific PCR. However, the simplicity and versatility of the combined PCR/ASO method has led to its continued use, including with non-radioactive labels, and in a “reverse dot blot” format where the ASO probes are bound to the membrane and the amplified sample DNA is used for hybridization.

Methods and materials of the invention may be used more generally to evaluate a DNA sample from a subject, genetically type the subject, and detect genetic differences between subjects. In one embodiment of the invention, a biological sample which includes DNA from a subject is evaluated to detect the genotype of the subject for a nucleotide that occurs at any of the SNPs labeled here as SNPs “1a” to “13m.” A sample of genomic DNA from a subject may be evaluated by reference to one or more controls (or normal) to determine if a SNP or group of SNPs is present. With this present invention, any method for determining genotype can be used for determining the genotype of the subject. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, microsatellite analysis, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties. Genotypic data useful in the methods of the invention and methods for the identification and selection of genes associated with CA-MRSA are based on the presence of SNPs.

Single Nucleotide Polymorphisms Associated with CA-MRSA

Genomic DNA was obtained from a population of patients with recurrent CA-MRSA as well as from healthy spouse controls, which were likely to have been exposed to the same MRSA bacteria as the recurrent CA-MRSA patients. A microarray hybridization assay for single nucleotide polymorphism (SNP) alleles that segregated between the CA-MRSA and control populations was performed that was capable of detecting the presence of 906,000 known polymorphisms as well as their copy number in each subject (i.e., that could detect, whether the subject had multiple gene copies, and whether the subject was homozygous or heterozygous for particular allele(s) at a particular locus.)

A highly segregated SNP was found in the FAM129B gene, where all CA-MRSA subjects tested had two copies (homozygous) of one allele and control subjects had two copies (homozygous) of another allele. This SNP is present at a loci corresponding to position 17460 of SEQ ID NOs 1 and 2, wherein each of SEQ ID NOs 1 and 2 identify an alternate oligonucleotide at that position. More specifically, SEQ ID NO 1 identifies an “A” (adenine) at position 17460 and SEQ ID NO 2 identifies a “C” (cytosine) at position 17460. This SNP is located in an intron of the FAM129B gene. SEQ ID NOs 3 and 4 generally correspond with positions 17444-17464 of SEQ ID NO 1, the full-length DNA sequence of the FAM129B gene.

Subjects with at least one or more copies of the allele corresponding to position 17460 of SEQ ID NO 1 are at increased risk of developing at developing MRSA and/or CA-MRSA, or a recurrence of MRSA or CA-MRSA. And subjects with at least one or more copies of the allele corresponding to position 17460 of SEQ ID NO 2 are not at increased risk of developing at developing MRSA and/or CA-MRSA, or a recurrence of MRSA or CA-MRSA.

The FAM129B gene encodes a protein that has a predicted molecular mass of 83 kDa, and contains a pleckstrin homology domain and a proline-rich region that contains six serine phosphorylation sites (Chen et al (2011) J. Biol. Chem. 286(12):10201-10209; Old et al. (2009) Mol. Cell 34: 115-131). Phosphorylation has been associated with MAP kinase signaling cascade; in melanoma cells the MAP kinase pathway was active and the FAM129B protein was localized throughout the cytoplasm. When the MAP kinase pathway was inhibited, the FAM129B protein migrated to the cell membrane and melanoma cell migration through a collagen matrix was inhibited. (Old et al., p. 125). Subsequent work found that FAM129B was cytoplasmically localized in actively growing HeLa cells, but appeared to be localized at cell-cell junctions on the plasma membrane when the HeLa cells achieved confluence, and throughout the cell membrane during telophase. (Chen et al. pp. 10203-10204.) FAM129B also inhibited apoptosis in HeLa cells treated with TNFα or CHX, compared with knockdown FAM129B HeLa cells silenced with siRNA sequences specific to FAM129B. A recent investigation of the corresponding Fam129B protein in mice showed that Fam129B is expressed in the epidermal keratinocytes in embryonic and adult mice. Fam129B-knockout mice exhibited delayed wound healing and had altered expression of several wound-repair and cell-motility related genes (Oishi et al. (published online Sep. 11, 2012), J. Biochem. doi:10.1093/jb/mvs100).

Using similar procedures as above critical SNPs “a1” to “m13” were also found, located and described as disclosed herein. While this experiment happened to use patients thought to have CA-MRSA we fully expect to see the same results with all forms of MRSA. While this experiment happened to use patients with homozygous alleles we fully expect the same results would apply to those with heterozygous alleles.

Methods of Diagnosing Increased Risk of Developing MRSA

Aspects of the present invention comprise methods of determining whether a subject is at increased risk of developing MRSA, HA-MRSA, CA-MRSA, or SSI, or a recurrence of any form of MRSA or SSI comprising: obtaining a biological sample from a subject; obtaining at least one oligonucleotide from said biological sample that contains a loci corresponding to position 17460 of SEQ ID NOs 1 and 2; detecting in the oligonucleotide the identity of a nucleotide that occurs at a loci corresponding to position 17460 of SEQ ID NOs 1 and 2; and comparing the identity of the nucleotide that occurs at a loci corresponding to position 17460 of SEQ ID NOs 1 and 2 in the oligonucleotide to the identity of a nucleotide at position 17 of SEQ ID NO 3 and/or SEQ ID NO 4, wherein the subject is at increased risk of developing MRSA and/or CA-MRSA, or a recurrence of MRSA or CA-MRSA if the nucleotide that occurs at the loci corresponding to position 17460 of SEQ ID NOs 1 and 2 in the oligonucleotide is the same as the identity of the nucleotide at position 17 of SEQ ID NO 4, and wherein the subject is not at increased risk of developing MRSA and/or CA-MRSA, or a recurrence of MRSA or CA-MRSA if the nucleotide that occurs at the loci corresponding to position 17 of SEQ ID NOs 3 and 4 in the oligonucleotide is the same as the identity of the nucleotide at position 17460 of SEQ ID NO 2.

We believe and describe for the purposes of this document that any single or combination of SNPs a1-m13, as described herein, can also be used to identify MRSA and SSI high risk individuals.

Obtaining Oligonucleotides from Patients and Subjects

Biological samples may be any material or fluid (blood, lymph, etc.) derived from the body of a subject, that contains or may contain genomic DNA (chromosomal and mitochondrial DNA) or other oligonucleotides such as, for example, mRNA that derive from genomic DNA, or an organ or tissue extract and culture fluid in which any cells or tissue preparation from a subject has been incubated. Methods of obtaining biological samples and methods of obtaining oligonucleotide molecules such as DNA and RNA from a biological sample are well known in the art, such as blood draws, cheek cell swabs, biopsies and the like.

For purposes of obtaining at least one oligonucleotide from said biological sample that contains a loci corresponding to position 17460 of SEQ ID NOs 1 and 2, or any single or combination of SNPs a1-m13; DNA or other oligonucleotides, such as pre-mRNA, can be extracted or partially purified from the biological sample for further processing by techniques known to those skilled in the art (see, e.g., U.S. Pat. Nos. 6,548,256 and 5,989,431; Hirota et al. (1989) Jinrui Idengaku Zasshi. 34: 217-23 and John et al. (1991) Nucleic Acids Res. 19:408, the disclosures of which are incorporated by reference in their entireties). For example, high molecular weight DNA may be purified from cells or tissue using proteinase K extraction and ethanol precipitation. DNA, however, may be extracted from an animal specimen using any other suitable methods known in the art.

Alternatively, a purification step may be not be needed where probes such as those described below may operate to detect the presence of a SNP by directly hybridizing to genomic DNA in situ in the biological sample, such that obtaining at least one oligonucleotide from said biological sample that contains a loci corresponding to position 17 of SEQ ID. NO 1 and 2, or any single or combination of SNPs a1-m13; may occur without an oligonucleotide extraction step from the biological sample. The biological sample may be partially processed (i.e., homogenization, partial purification) prior to hybridization to facilitate the hybridization step.

Detecting SNP Polymorphisms

Any method of detecting the identity of individual nucleotides at SNP loci may be used to practice this invention.

In one aspect, detecting the identity of the SNP corresponding to position 17460 of SEQ ID NOs 1 and 2; or any single SNP or any combination of SNPs a1-m13, of the present invention, may be performed by sequencing the region of the genomic DNA sample that spans the identified polymorphic locus or SNPs 1a to m13. Many methods of sequencing genomic DNA are known in the art, and any such method can be used, see for example, Sambrook et al. (2001) Molecular Cloning: A Laboratory Manual, 3rd ed., Cold Spring Harbor Press. For instance, as described below, a DNA restriction fragment spanning the location of the SNP of interest can be amplified using the polymerase chain reaction, then subjected to further genomic sequencing methods.

In other aspects, detecting the identity of the SNP corresponding to position 17460 of SEQ ID NOs 1 and 2; or any single or combination of SNPs a1-m13 of the present invention may be performed by the use of allele-specific probes that hybridize to a region of DNA containing the allele of interest. The probes may be further tagged with a detection signal to aid in detecting the presence of the allele in the biological sample. Probes and detection signals are described below.

Amplification

A genomic oligonucleotide spanning the location of the SNP of interest in the FAM129B gene may also be amplified as part of the detection step. More specifically, detecting the identity of SNP of the present invention may comprise DNA amplification to amplify specific genomic sequences or regions containing the SNP correlated to healthy and/or recurrent CA-MRSA subject phenotypes, by one of several known methods of DNA amplification, such as PCR. As noted above, the PCR amplification process involves a cyclic enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The methods of the present invention may use oligonucleotide primers to amplify specific, genomic sequences containing the SNP correlated to healthy and/or recurrent CA-MRSA subject phenotypes. Such primers should be of sufficient length to enable specific annealing or hybridization to the nucleic acid sample. The sequences typically will be about 8 to about 44 nucleotides in length, but shorter or longer sequences may be used. Sequences from about 14 to about 50, may be advantageous for certain embodiments. The design of primers is well known to one of ordinary skill in the art. Primers may comprise sequences upstream or downstream of the location of the SNP, but not contain the SNP itself (begin or end at, e.g., 5-1000 base pairs upstream or downstream of the location of the SNP), or comprise a sequence comprising the SNP. Such primers may be used to specifically amplify one allele or another at that SNP location. In any case, primers should be designed such that the SNP is contained within the amplified sequence. For instance, suitable primers may be designed using sequences within SEQ ID NO 1 (the FAM129B gene) upstream or downstream from the location of the SNP at position 17 of SEQ ID NOs 3 and 4; or any single or combination of SNPs a1-m13, and primers upstream or downstream from the location of the SNP.

Where it is desired to amplify a fragment of DNA that comprises a SNP according to the present invention, the forward and reverse primers may have contiguous stretches of about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or any other length from 5 to 1500 nucleotides in length, with preferred lengths of 8 to 30 sequences and typically less than 50, 100 or 150 or less. The sequences to which the forward and reverse primers anneal are advantageously located on either side of the particular nucleotide position that is substituted in the SNP to be amplified (e.g., position 17 of SEQ ID NOs 3 and 4); or including any single or combination of the positions of SNPs a1-m13.

Oligonucleotide primers can be produced by a conventional production process for general oligonucleotides. They can be produced, for example, by a chemical synthesis process or by a microbial process that makes use of a plasmid vector, a phage vector or the like. Further, it is suitable to use a nucleic acid synthesizer.

Oligonucleotide Sequencing.

As noted above, detecting the identity of the SNP corresponding to position 17 of SEQ ID NOs 3 and 4 of the present invention may be performed by sequencing the region of the genomic DNA sample that spans the FAM 129B polymorphic locus, or any of the SNP positions as described as SNP 1a to m13. Reagents allowing the sequencing of reaction products can be utilized herein. For example, chain-terminating nucleotides will often be incorporated into a reaction product during one or more cycles of a reaction. Commercial kits containing the reagents most typically used for these methods of DNA sequencing are available and widely used. PCR exonuclease digestion methods for DNA sequencing can also be used. Many methods of sequencing genomic DNA are known in the art, and any such method can be used, see for example Sambrook et al. (2001) Molecular Cloning: A Laboratory Manual, 3rd ed., Cold Spring Harbor Press. For example, as described below, a DNA fragment spanning the location of the SNP of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, (2000) Genome Res. 10: 1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, (2001) Methods Mol. Biol. 167: 153-70 and MacBeath et al. (2001) Methods Mol. Biol. 167:119-52), capillary electrophoresis (see, e.g., Bosserhoff et al. (2000) Comb Chem High Throughput Screen. 3: 455-66), DNA sequencing chips (see, e.g., Jain, (2000) Pharmacogenomics. 1: 289-307), mass spectrometry (see, e.g., Yates, (2000) Trends Genet. 16: 5-8), pyrosequencing (see, e.g., Ronaghi, (2001) Genome Res. 11: 3-11), and ultrathin-layer gel electrophoresis (see, e.g., Guttman & Ronai, (2000) Electrophoresis. 21: 3952-64), the disclosures of which are hereby incorporated by reference in their entireties. The sequencing can also be done by a commercial company. Examples of such companies include, but are not limited to, the University of Georgia Molecular Genetics Instrumentation Facility (Athens, Ga.) or SeqWright DNA Technologies Services (Houston, Tex.).

Oligonucleotide Hybridization

Detecting the identity of a SNP corresponding to position 17 of SEQ ID NOs 3 and 4 of the present invention, and the SNPs corresponding to the positions provided and described as SNPs “1a” to “13m,” may be performed by the use of allele-specific probes that hybridize to a region of DNA containing the allele of interest.

One example method for determining the genotype at the polymorphic locus encompasses obtaining a biological sample that includes a nucleic acid sample, hybridizing the nucleic acid sample with a probe, and disrupting the hybridization to determine the level of disruption energy required wherein the probe has a different disruption energy for one allele as compared to another allele. In one example, there can be a lower disruption energy, e.g., melting temperature, for an allele that harbors a cytosine residue at a polymorphic locus, and a higher required energy for an allele with a different residue at that polymorphic locus. This can be achieved where the probe has 100% sequence identity with one allele (a perfectly matched probe), but has a single mismatch with the alternative allele. Since the perfectly matched probe is bound more tightly to the target DNA than the mismatched probe, it requires more energy to cause the hybridized probe to dissociate.

In a further step of the above method, a second (“anchor”) probe may be used. Generally, the anchor probe is not specific to either allele, but hybridizes regardless of what nucleotide is present at the polymorphic locus. The anchor probe does not affect the disruption energy required to disassociate the hybridization complex but, instead, contains a complementary label for using with the first (“sensor”) probe.

Hybridization stability may be influenced by numerous factors, including thermoregulation, chemical regulation, as well as electronic stringency control, either alone or in combination with the other listed factors. Through the use of stringency conditions, in either or both of the target hybridization step or the sensor oligonucleotide stringency step, rapid completion of the process may be achieved. This is desirable to achieve properly indexed hybridization of the target DNA to attain the maximum number of molecules at a test site with an accurate hybridization complex. By way of example, with the use of stringency, the initial hybridization step may be completed in ten minutes or less, more advantageously five minutes or less, and most advantageously two minutes or less. Overall, the analytical process may be completed in less than half an hour.

In one mode, the hybridization complex is labeled and the step of determining the amount of hybridization includes detecting the amounts of labeled hybridization complex at the test sites. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. The labeled portion of the complex may be the target, the stabilizer, the probe or the hybridization complex in toto. Labeling may be by fluorescent labeling selected from the group of, but not limited to, Cy3, Cy5, Bodipy Texas Red, Bodipy Far Red, Lucifer Yellow, Bodipy 630/650-X, Bodipy R6G-X and 5-CR 6G. Colorimetric labeling, bioluminescent labeling and/or chemiluminescent labeling may further accomplish labeling. Labeling further may include energy transfer between molecules in the hybridization complex by perturbation analysis, quenching, electron transport between donor and acceptor molecules, the latter of which may be facilitated by double stranded match hybridization complexes. Optionally, if the hybridization complex is unlabeled, detection may be accomplished by measurement of conductance differential between double stranded and non-double stranded DNA. Further, direct detection may be achieved by porous silicon-based optical interferometry or by mass spectrometry. In using mass spectrometry no fluorescent or other label is necessary. Rather detection is obtained by extremely high levels of mass resolution achieved by direct measurement, for example, by time of flight (TOF) or by electron spray ionization (ESI). Where mass spectrometry is contemplated, probes having a nucleic acid sequence of 50 bases or less are advantageous.

The label may be amplified, and may include, for example, branched or dendritic DNA. If the target DNA is purified, it may be un-amplified or amplified. Further, if the purified target is amplified and the amplification is an exponential method, it may be, for example, PCR amplified DNA or strand displacement amplification (SDA) amplified DNA. Linear methods of DNA amplification such as rolling circle or transcriptional runoff may also be used.

A detectable label can be incorporated into a nucleic acid during at least one cycle of an amplification reaction. Spectroscopic, photochemical, biochemical, immunochemical, electrical, optical or chemical means can detect such labels. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, ³²P, etc.), enzymes (e.g., horseradish peroxidase, alkaline phosphatase etc.), calorimetric labels such as colloidal gold or colored glass or plastic (e.g., polystyrene, polypropylene, latex, etc.) beads. The label is coupled directly or indirectly to a component of the assay according to methods well known in the art. As indicated above, a wide variety of labels are used, with the choice of label depending on sensitivity required, ease of conjugation with the compound, stability requirements, available instrumentation, and disposal provisions. Non-radioactive labels are often attached by indirect means. Polymerases can also incorporate fluorescent nucleotides during synthesis of nucleic acids.

To label an oligonucleotide with the fluorescent dye, one of several conventionally known labeling methods can be used (Tyagi & Kramer (1996) Nature Biotechnology 14: 303-308; Schofield et al. (1997) Appl. and Environ. Microbiol. 63: 1143-1147; Proudnikov & Mirzabekov (1996) Nucl. Acids Res. 24: 4532-4535). Alternatively, the oligonucleotide may be labeled with a radiolabel e.g., ³H, ¹²⁵I, ³⁵S, ¹⁴C, ³²P, etc. Well-known labeling methods are described, for example, in Sambrook et al. (2001) Molecular Cloning: A Laboratory Manual, 3rd ed., Cold Spring Harbor Press. The label is coupled directly or indirectly to a component of the oligonucleotide according to methods well known in the art. Reversed phase chromatography or the like used to provide a nucleic acid probe for use in the present invention can purify the synthesized oligonucleotide labeled with a marker. An advantageous probe form is one labeled with a fluorescent dye at the 3′- or 5′-end and containing G or C as the base at the labeled end. If the 5′-end is labeled and the 3′-end is not labeled, the OH group on the C atom at the 3′-position of the 3′-end ribose or deoxyribose may be modified with a phosphate group or the like although no limitation is imposed in this respect.

During the hybridization of the nucleic acid target with the probes, stringent conditions may be utilized, advantageously along with other stringency affecting conditions, to aid in the hybridization. Detection by differential disruption is particularly advantageous to reduce or eliminate slippage hybridization among probes and target, and to promote more effective hybridization. In yet another aspect, stringency conditions may be varied during the hybridization complex stability determination so as to more accurately or quickly determine whether a SNP is present in the target sequence.

A SNP-specific probe can also be used in the detection of the SNP in amplified specific nucleic acid sequences of the target gene FAM129B, or any single or combination of SNPs a1-m13, such as the amplified PCR products generated using the primers described above. In certain embodiments, these SNP-specific probes consist of oligonucleotide fragments. Advantageously, the fragments are of sufficient length to provide specific hybridization to the nucleic acid sample. The use of a hybridization probe of between 10 and 50 nucleotides in length allows the formation of a duplex molecule that is both stable and selective. Longer probes may be used. Molecules having complementary sequences over stretches greater than 12 bases in length are generally advantageous, in order to increase stability and selectivity of the hybrid, and thereby improve the quality and degree of particular hybrid molecules obtained. One will generally prefer to design nucleic acid molecules having stretches of 16 to 24 nucleotides, or even longer where desired. A tag nucleotide region may be included, as at the 5′ end of the primer that may provide a site to which an oligonucleotide sequencing primer may hybridize to facilitate the sequencing of multiple PCR samples.

The probe sequence must span the particular nucleotide position that may be substituted in the particular SNP to be detected, here, the positions provided for SNPs 1a to m13, such as position 17 of SEQ ID NOs 3 and 4 for SNP 1a. Advantageously, two or more different “allele-specific probes” may be used for analysis of a SNP, a first allele-specific probe for detection of one allele, and a second allele-specific probe for the detection of the alternative allele. For example, one probe could be used for detection of the adenosine at position 17 of SEQ ID NO. 3 and another probe could be used for detection of cytosine at position 17 of SEQ ID NO. 4, or any single or combination of SNPs a1-m13.

It will be understood that this invention is not limited to the particular primers and probes disclosed herein and is intended to encompass at least nucleic acid sequences that are hybridizable to the nucleotide sequence disclosed herein, the complements or any suitable fragment thereof, or to functional sequence analogs of these sequences. Homologs (i.e., nucleic acids derived from other species) or other related sequences (e.g., paralogs) can be obtained under conditions of standard or stringent hybridization conditions with all or a portion of the particular sequence as a probe using methods well known in the art for nucleic acid hybridization and cloning.

Advantageously, probes may be affixed to substrates and used in “microarray” and other high-throughput detection applications such as those used in the Example below and which are well known in the art. Microarrays can show the presence of one or both SNP alleles, copy number (such as whether an individual is homozygotic, or heterozygotic for a particular polymorphism), and thus provide a genotype for an individual subject.

Subjects at Increased Risk of MRSA

In one embodiment of the present invention, subjects with at least one or more copies of the allele corresponding to position 17 of SEQ ID NO 3 are at increased risk of developing MRSA and/or HA or CA-MRSA, or a recurrence of MRSA or HA or CA-MRSA. In another embodiment of the present invention, subjects with at least one or more copies of the allele corresponding to position 17 of SEQ ID NO 4 are not at increased risk of developing at developing MRSA and/or CA-MRSA, or a recurrence of MRSA or CA-MRSA. This analysis of the SNP we name “a1” herein can also be used with other appropriate sequences with all the other critical SNPs described herein as a1 to m13 and any combinations thereof.

Other SNPs, or other biomarkers, such as gene or protein biomarker, miRNA and the like, the levels or presence/absence of which are correlated with increased risk of developing MRSA or HA or CA-MRSA, or the occurrence of recurrent MRSA or HA or CA-MRSA may also be used alone or in conjunction with the SNP of the present invention to diagnose subjects who are at increased risk of developing MRSA or HA or and/or CA-MRSA, or a recurrence of or HA or MRSA or CA-MRSA.

Other SNPs or biomarkers may identify a structurally or functionally abnormal FAM129B gene caused by a point mutation(s), a deletion, a truncation, or a translocation of at least a portion of the FAM129B gene, or the other genes or other inter gene regions we describe herein. An exemplary biomarker may identify and/or detect the presence of (a) a decrease or an increase in expression of the FAM129B gene (as compared to a control group which is not at an increased risk of developing MRSA or CA-MRSA, or recurrent MRSA or CA-MRSA) or (b) the abnormal methylation of at least a part of the FAM129B gene. In one embodiment, methods to detect a structurally or functionally abnormal FAM129B gene may include using an oligonucleotide primer that is complementary to or identical to a portion of SEQ ID NO 3 to amplify an oligonucleotide sample from a subject (and the amplified oligonucleotides may then be sequenced); or hybridizing oligonucleotides in a sample from a subject to an oligonucleotide probe having a sequence that is complementary to or identical to a portion of SEQ ID NO 3.

In many cases a patient may exhibit more than one SNP that indicates a greater than normal risk of acquiring a MRSA or SSI infection. The discovery of more than one MRSA indicating SNP base in a person is not always indicative of a greater chance of becoming infected than if only one SNP base is found. In some cases the greater the number of different MRSA high risk SNPs an individual has, the greater confidence one can have that the individual is at high risk of developing a MRSA infection following exposure to the MRSA bacteria. In other cases just one, or any combination of 2 or more alleles with provide great confidence that the individual is at high risk of developing a MRSA infection following exposure to the MRSA bacteria.

Treatment of Patients at Increased Risk of MRSA and or Recurrent MRSA

In some aspects of the method, subjects found to be at increased risk of developing MRSA, HA-MRSA, CA-MRSA, SSI or having a recurrence of MRSA, HA-MRSA, CA-MRSA or SSI may be treated with an antibiotic effective against MRSA. In other aspects, subjects found to be at increased risk of developing MRSA or having a recurrence of MRSA can be treated with appropriate topical and/or nasal treatments to remove surface colonies of or prevent colonization by skin-surface or intranasal populations of MRSA. Appropriate treatments may be increased sanitation (more frequent hand washing with regular or antibiotic soaps such as Hibiclens (4% clorhexidine), topical antibiotic treatments, and oral antibiotics, mouth rinses and nasal ointments containing antibiotics. See, e.g., Buehlmann, M. et al. “Highly effective regimen for decolonization of methicillin-resistant Staphylococcus aureus carriers” Infect. Control. Hosp. Epidemiol. (2008) 29(8); 510-6.

A more aggressive treatment of a MRSA, HA-MRSA, CA-MRSA or SSI patient may involve the administration of an antibiotic regime including the repeated and/or prophylactic use of one or more anti-MRSA antibiotics such as Vancomycin, Daptomycin, Linezolid, Ceftaroline, Telavancin, Bactrim and the like. Treatment of MRSA patients often includes decolonization efforts, frequent monitoring, long term follow-up and special treatment for any further surgeries (e.g. surgical prescreening for MRSA and antibiotic treatment for prophylaxis) including long duration evaluation and monitoring for infection. Treatment for patients at normal risk of MRSA infection could involve as little as incision and drainage followed by administration of a common antibiotic or in some cases no antibiotic at all.

Kits

Kits comprising the methods and devices to determine the MRSA risk assessment are described here and methods to make and use such kits, using the information and descriptions provided herein would be known to one skilled in the art given the descriptions provided.

Some embodiments of the present invention comprise a kit for determining whether a subject is at increased risk of developing MRSA, or a recurrence of MRSA comprising at least one primer for amplification of one or more nucleotides that occur at a loci corresponding to position 17 of comparison SEQ ID NOs 3, 4, and the SNPs a1-m13 described herein or any combination thereof from a biological sample from the subject.

Other embodiments of the present invention comprise a kit for determining whether a subject is at increased risk of developing MRSA or SSI comprising at least one probe for detection of one or more nucleotides that occur at a loci corresponding to the loci and positions of SNPs a1-m13 described herein or any combination thereof from a biological sample from the subject. For example for SNP a1, one would create a kit to probe position 17 of comparison SEQ ID NOs 3 and 4 of the FAM 129 B gene.

Some embodiments of the invention may comprise one or more probes for use in determining whether a subject is at increased risk of developing MRSA, CA-MRSA, HA-MRSA, SSI or a recurrence of MRSA, CA-MRSA, HA-MRSA or SSI wherein the probe or probes comprise the oligonucleotide(s) described by SNP a1 using portions of SEQ ID NOs 3 and/or 4; and the nucleotides for control or normal and MRSA or SSI high risk, such as are described by SNPs a1-m13 described herein or any combination thereof. Other embodiments of the invention may comprise an amplification product for use in determining whether a subject is at increased risk of developing MRSA, CA-MRSA, HA-MRSA, SSI or a recurrence of MRSA, CA-MRSA, HA-MRSA, or SSI wherein the amplification product comprises an oligonucleotide sequence comprising SEQ ID NOs 3 and/or 4; and the SNPs a1-m13 described herein or any combination thereof. Other embodiments of the invention may comprise amplification primers for use in determining whether a subject is at increased risk of developing MRSA, CA-MRSA, HA-MRSA, SSI or a recurrence of MRSA, CA-MRSA, MA-MRSA or SSI wherein the amplification primers comprise oligonucleotide sequences in any of the sequences in the Sequence listing, including SEQ ID NOs 1-37, and those oligonucleotide immediately flanking the location of the SNPs a1 to m13, and critical SNP positions identified in for SNPs a1 to m13 and from SEQ ID NOs 1-37: comprising SEQ ID NOs 1-37 and oligonucleotide sequences in SEQ ID NOs 1-37 and immediately flanking the location of the critical SNP positions of SEQ ID NOs 1-37 or comprise SEQ ID NOs 1-37. And for all of these situations and uses including all single and combinations of all oligonucleotides sequences identifiable from SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37, and every possible combination of those SNPs and genes.

Risk Profile

Clinically, a biomarker panel for risk of MRSA infection, here Risk Profile, will allow for a more aggressive approach in treatment for patients who are at high risk for MRSA infections and especially for patients who develop their first MRSA infection. Currently these patients go unrecognized until they have several infections before more aggressive treatment regimens are prescribed in order to eradicate the MRSA from their skin. Being able to determine what patients would benefit from, i.e., those who need early aggressive treatment to reduce recurrences, readmissions, and morbidity would be of substantial benefit.

Similarly, being able to determine a subset of surgical patients in whom aggressive post-surgical prophylactic therapy would prevent infections would be of tremendous value and reduce complication rates, readmissions, transmission and infection of others, length of stay, as well of overall cost and morbidity and mortality. Such a test or diagnostic with appropriate treatments or conditions provided could become the standard of care for all patients who are to undergo surgery. Since the test determines genetic risk for infection, it would only need to be run once, ever, for any patient, for his or her lifetime. It could be used for patients who have never before been exposed to MRSA and thus those whose susceptibility to SSI and MRSA and related staph infections are unknown. It could be used to distinguish and accurately diagnose those patients who have been incorrectly diagnosed as being at high risk of getting a MRSA infection but who in fact are normal and not at high risk. It could be used to confirm those who have had one or two previous MRSA infections are indeed at high risk of getting another MRSA infection and who have the greatest need of receiving the aggressive treatments described herein and known to those skilled in the art of prescribing and giving aggressive MRSA and SSI treatments.

Risk Profiling from the use of Biomarker Panels coupled with appropriate treatment. Risk Profiling can involve both the evaluation and detection of biomarkers, the generic factors and also the evaluation of traditional risk factors that may also be coupled with treatment. These two types of risk factors may be created and used independently or they may be combined together and or combined with other factors.

The following types of information are typically collected, assessed and analyzed in order to make biomarker panels and perform risk profiling.

Part 1.

The Genetic Risk Profile is a summary of the number of, and analysis of, the critical SNP positions and critical SNP nucleotides as measured and detected in the patient. This may include: the total number of MRSA high risk SNPs and nucleotides with a description of those SNPs and MRSA high risk genes involved, for the described SNPs, both normal and the same information for MRSA low risk SNPs, with a summary of those measurements and results for some or all of the described critical SNPs at the critical SNP positions, from a1 to m13 as described herein and their nucleotides and the characterization of those nucleotides as high or normal risk, including SNPs from a1 to m13 as described herein, and any combination thereof, which are the factors collectively described here as the “genetic factors.”

Part 2.

Physical MRSA risk factors are based on knowledge of the patient and the patient's medical history; most physical risk factors are known or will be learned by caregivers skilled in the art. The list of physical MRSA here is intended only to provide examples rather than provide an exhaustive list. Traditional risk factors are often collected from patients during intake, from pre surgical evaluations and questions, from the settings past and present of the patient, from interviews with the patient, the patient's medical history, the patient's family or from those who know the patient. The questions might include some or all of the following.

Has the patient or members of their family ever previously been told they may have or did they ever previously have MRSA?

Has the patient or members of their family ever previously been told they may have or did they ever previously have a staph infection?

Has the patient or members of their family ever previously been told they may have or did they ever previously have a persistent skin infection?

Has the patient or members of their family ever previously been told they may have or did they ever previously have any of the following:

administration of multiple antibiotics to treat a single infection,

ulcers and pressure ulcers, surgical wounds,

nasogastric or endotracheal tubes, drains, and urinary or intravenous catheterization,

admission into intensive care.

Were those having the following given an increased risk assessment: the people who were preciously admitted into intensive care, those that have had surgical wounds, pressure ulcers, and or intravenous catheterization.

Has the patient or members of their family ever previously been told they are at risk of developing MRSA following surgery?

Among the more important physical risk factors are: prior admittance into intensive care, those that have had surgical wounds, pressure ulcers, and or intravenous catheterization.

Part 3. The Overall Risk Profile.

The Overall Risk Profile for a patient is any combination of the genetic and physical risk factors. Various weights of importance can be given to the risk factors by the treating caregiver and appropriate action or inaction taken. The generic risk factors may be considered or in combination with the physical risk factors.

The Overall Risk Profile is created by assigning values to both the genetic and traditional risk factors and for both MRSA patients, or “high risk” patients, and “normal” or average risk patients. Treatment decisions may be made depending on the various risk factors.

### Examples

Study Design.

Fourteen participants were contacted and consented to collection of blood samples for analysis. Collection and analysis were approved through the Beaumont Institutional Review Board. Eleven participants were patients who had proven history of and were often seen for recurrent CA-MRSA but had no known specific risk factors for developing recurrent infection. Three participants were controls and were cohabiting spouses of three of the patients. The controls were exposed to the same environmental risks as the patients and to the skin of the patient but never developed a MRSA infection. This gave controls who were directly and closely exposed to the patient (i.e., shared a bed) and were thus within the same environment but did not become infected with MRSA.

Methods.

Collection and analysis was done via the Beaumont BioBank. DNA was isolated from blood samples of the MRSA-infected patients, the associated controls, and a subset of the SSI patients. The DNA was used for preparation of DNA libraries. Library prep involved fragmenting the original DNA, subsequent processing to add primers and adaptors, and finally enriching for the regions of interest. Probes for the enrichment process were designed through Illumina's Design Studio using Nextera Rapid Capture Custom Enrichment. This process allows for the design of enrichment probes that cover the entire coding and non-coding sequence of any gene of interest. Once libraries were created, they were sequenced on the Illumina NextSeq 500. The resulting data files were uploaded to Illumina BaseSpace for de-multiplexing and file conversion. Alignment was done with NextGENe software from Soft Genetics, with additional analysis done with Ingenuity Variant Analysis.

Analysis was performed in an automated and blinded manner. Genomic DNA from all participants was prepared for analysis using Affymetrix Genome-wide Human SNP 6.0 microarrays. Each array contains more than 946,000 probes for detection of copy number variation and more than 906,000 single nucleotide polymorphism (SNP) probes for genotyping. One array per patient sample was prepared according to the manufacturer's protocol and scanned with an Affymetrix GeneChip® Scanner 3000. Affymetrix Genotyping Consol software and the Partek Genomics Suite were used for analysis and visualization of the data.

Data was subjected to per SNP and per sample quality control to minimize false positives. None of the remaining samples from individuals were excluded based on the expression data. SNPs from X and Y chromosomes were excluded from further analysis. SNPs with no call rates <5% and minor alleles frequencies >5% were included for further analysis. The final number of SNPs included in the analysis was 633,268.

A chi-square test was used to set the phenotype to be tested for association with the SNPs. Three models were tested:

1. Allele: frequencies of alleles (A vs. A′) were compared between CA-MRSA subjects and the control subjects
2. Genotype: frequencies of three possible genotypes (AA, AA′, and A′A′) were compared between CA-MRSA subjects and the control subjects
3. Dominant/Recessive: two combinations of genotypes are compared between CA-MRSA subjects and the control subjects—Dominant (AA+AK vs. A′A′, with A as the causal variant) and Recessive (AA vs. AA′+A′A′, with A as the causal variant).

In general we mention that one of the more significant SNPs of interest, (p value 1.21×10-7), was located within the open reading frame of a gene identified as FAM129B. This SNP (SNP_A 8307872, rs2249861) was present with two copies of a single form in all 11 MRSA patients and with two copies of another form in all three controls. There were no participants who were heterozygous (one copy of the gene in each form).

The particular SNP in gene FAM129B which segregated between the control and CA-MRSA populations was located in an intron sequence. The SNP has the following sequence in CA-MRSA subjects: GGGGGCAAGTTAGTCAACCTGTCTGAGTCTTAG [SEQ ID NO 3] with the SNP location at position 17 underlined. Control populations had the alternate allele: GGGGGCAAGTTAGTCACCCTGTCTGAGTCTTAG [SEQ ID NO 4] at position 17.

The FAM129B gene codes for a protein found in the tight junctions of skin cells and has been linked to wound healing and the ability of melanoma to invade the skin 2-4.

Other SNPs of interest are as follows:

The location of the SNPs in the genome. Most of the SNPs we describe are within genes but one SNP is between two genes. The SNPs or markers we describe are in or near the following genes: FAM 129B (1 SNP), RBM6 (1 SNP), KANK 4 (1 SNP), Col 13A1 (1 SNP), COL 19A1 (5 SNPs), IGSF8 (1 SNP), ADARB2 (1 SNP), DCT (1 SNP), and the last SNP lies between genes LTF and CCRL-2. The number in parenthesis is the number of SNPs associated with that gene.

The SNPs in this document are described with a unique alpha numeric number and those SNP numbers and the closest genes are:

“a1” is known as SNP/rs2249861 and it is within the FAM 129B gene;
“b2” is known as SNP/rs2352969 and it is within the RBM6 gene;
“c3” is known as SNP/rs12758910 and it is within the KANK 4 gene;
“d4” is known as SNP/rs2642572 and it is outside and between two genes, the C10orf35 isoform X1 gene and the Col 13A1 gene;
“e5” is known as SNP/rs3793038 and it is within the Col 19A1 gene;
“f6” is known as SNP/rs3805987 and it is within the Col 19A1 gene;
“g7” is known as SNP/rs9454944 and it is within the Col 19A1 gene;
“h8” is known as SNP/rs978291 and it is within the Col 19A1 gene;
“i9” is known as SNP/rs978290 and it is within the Col 19A1 gene;
“j10” is known as SNP/rs1131891 and it is within the IGSF8 gene;
“k11” is known as SNP/rs127711411 and it is within the ADARB2 gene;
“l12” is known as SNP/rs4773794 and it is within the DCT gene; and
“m13” is known as SNP/rs4619820 and it is outside and between two genes, CCR2 and LTF.

The genes are known for the following traits. The chromosomes where the SNPs are located, the sequence information, the useful probes and genetic information are provided above and in the sequence listing.

IGFS8, is the immunoglobulin superfamily protein 8, is linked to cell motility and polarity, cell adhesion, migration of leukocytes between cells, and infection with parasites and viruses.

ADARB2, adenosine deaminase RNA-specific B2, is linked to neurologic diseases.

DCT, dopachrome tautomerase, is found in skin cells and is melanogenic. The DCT-phenotype is associated with enhanced neurotization in benign nevi and with ulceration in thin malignant melanomas. DCT is a mediator of a melanoma stress-resistant pathway, and an antiapoptotic molecule.

KANK 4 is a homologue of genes involved in the regulation of actin polymerization and cell motility.

LCT, lactoferrin, stimulates the proliferation and migration of fibroblasts and keratinocytes and enhances the synthesis of extracellular matrix components, such as collagen and hyaluronan.

RBM6, RNA Binding Motif 6, is a tumor suppressor gene, promotes cell growth, and thus may potentially be involved in wound healing though this hasn't been studied.

Col 13A1 and Col 19A1 are collagens and thus a major component of the skin. Most of these genes have some role to play in the skin or wound healing.

What is claimed is:

1. A method of detecting whether a nucleotide of one or more single nucleotide polymorphisms (SNP) is present at a SNP position, wherein the one or more SNPs is selected from adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13, comprising:
obtaining from a human subject a blood sample having or suspected of having the nucleotide at the SNP position; and
detecting in the blood sample whether the nucleotide of the SNP is present at the SNP position, wherein the nucleotide is selected from cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and guanine at SNP position m13.

2. The method of claim 1, further comprising, detecting in the blood sample whether adenine is present at SNP position a1.

3. The method of claim 1, wherein the nucleotide is detected in two or more SNPs.

4. The method of claim 1 or 2, wherein the subject potentially has been exposed to or has been exposed to infection by antibiotic-resistant Staphylococcus aureus (MRSA) or a surgical site infection (SSI).

5. The method of claim 4, wherein (a) the subject has had a surgical procedure or a procedure involving an invasive medical device, has been living in unsanitary conditions, or has been participating in sports activities with exposure to cuts or other injuries; or (b) is in need of a surgery or a stay at a medical treatment or care facility.

6. The method of claim 1, wherein the detecting step includes contacting the blood sample with an oligonucleotide probe that is hybridizable with the nucleotide located at the SNP position, and detecting the hybridization.

7. The method of claim 1 or 2, further comprising: administering to the subject an aggressive treatment for recurrent MRSA when the detected nucleotide of the SNP is present at the SNP position.

8. The method of claim 7, wherein the aggressive treatment is selected from decolonization of MRSA from the skin or nasal passages of the subject, aggressive antibiotic therapy, administration of antibiotic-resistant drugs, isolation therapy, and recolonization therapy.

9. A method of detecting whether a nucleotide of one or more single nucleotide polymorphisms (SNP) is present at a SNP position, wherein the one or more SNPs is selected from adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13, comprising:
obtaining from a human patient a blood sample having or suspected of having the nucleotide at the SNP position; and
detecting in the blood sample whether the nucleotide of the SNP is present at the SNP position, wherein the nucleotide is selected from guanine at SNP position b2,
thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and adenine at SNP position m13.

10. The method of claim 9, further comprising, detecting in the blood sample whether cytosine is present at SNP position a1.

11. The method of claim 9 or 10, further comprising: not administering to the subject an aggressive treatment for recurrent MRSA when the detected nucleotide of the SNP is present at the SNP position.

12. A method of providing medical services to a human subject suspected of having or having recurrent antibiotic-resistant Staphylococcus aureus (MRSA) or a surgical site infection (SSI), comprising:
obtaining a biological sample from the subject;
requesting diagnostic information about the sample, wherein the diagnostic information is an identity of a nucleotide of one or more single nucleotide polymorphisms (SNP) at a SNP position, wherein the one or more SNPs is selected from adenine or cytosine at a SNP position a1, cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13; and
administering a treatment for recurrent MRSA or SSI when the identity of the nucleotide of one or more SNPs is a high MRSA risk SNP.

13. The method of claim 12, further comprising requesting additional diagnostic information about the sample, wherein the additional diagnostic information is an identity of adenine or cytosine at a SNP position a1; and administering a treatment for recurrent MRSA or SSI when the identity of the nucleotide of one or more SNPs is a high MRSA risk SNP and adenine is identified at the SNP position a1.

14. The method of claim 12, wherein the identity of the nucleotide of one or more SNPs is selected from cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and guanine at SNP position m13; and the treatment for recurrent MRSA or SSI is administered.

15. The method of claim 13, wherein the identity of the nucleotide at SNP position a1 is adenine; and the treatment for recurrent MRSA or SSI is administered.

16. The method of claim 14 or 15, wherein the treatment is selected from decolonization of MRSA from the skin or nasal passages of the subject, aggressive antibiotic therapy, administration of antibiotic-resistant drugs, isolation therapy, and recolonization therapy.

17. The method of claim 12, wherein the identity of the nucleotide of one or more SNPs is selected from guanine at SNP position b2, thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and adenine at SNP position m13; and the treatment for recurrent MRSA or SSI is not administered.

18. The method of claim 13, wherein the identity of the nucleotide at SNP position a1 is cytosine; and the treatment for recurrent MRSA or SSI is not administered.

19. An artificial probe comprising:
one or more oligonucleotides from 5 to 1500 nucleotides in length, and conjugated to a label, marker or detectable moiety,
wherein the one or more oligonucleotides is able to hybridize with from 5 to 1500 nucleotides adjacent to and including a single nucleotide located at a SNP position, and
wherein the single nucleotide is selected from the group consisting of cytosine or guanine at SNP position b2, guanine or thymine at SNP position c3, cytosine or thymine at SNP position d4, thymine or guanine at SNP position e5, guanine or adenine at SNP position f6, guanine or adenine at SNP position g7, thymine or cytosine at SNP position h8, adenine or guanine at SNP position i9, guanine or adenine at SNP position j10, adenine or guanine at SNP position k11, cytosine or thymine at SNP position l12, and guanine or adenine at SNP position m13.

20. The probe of claim 19, further comprising an oligonucleotide from 5 to 1500 nucleotides in length, and conjugated to a label, marker or detectable moiety, wherein the oligonucleotide is able to hybridize with from 5 to 1500 nucleotides adjacent to and including an adenine or cytosine located at a SNP position a1.

21. The probe of claim 19, wherein the one or more oligonucleotides is from 10 to 200 nucleotides.

22. The probe of claim 19, wherein the single nucleotide located at the SNP position is selected from the group consisting of: cytosine at SNP position b2, guanine at SNP position c3, cytosine at SNP position d4, thymine at SNP position e5, guanine at SNP position f6, guanine at SNP position g7, thymine at SNP position h8, adenine at SNP position i9, guanine at SNP position j10, adenine at SNP position k11, cytosine at SNP position l12, and guanine at SNP position m13.

23. The probe of claim 19, wherein the single nucleotide located at the SNP position is selected from the group consisting of guanine at SNP position b2, thymine at SNP position c3, thymine at SNP position d4, guanine at SNP position e5, adenine at SNP position f6, adenine at SNP position g7, cytosine at SNP position h8, guanine at SNP position i9, adenine at SNP position j10, guanine at SNP position k11, thymine at SNP position l12, and adenine at SNP position m13.

24. The probe of claim 19, wherein the one or more oligonucleotides is conjugated to a detectable moiety and the detectable moiety is selected from:
a) a radioactive label;
b) a P³² label; and
c) a fluorescent label.

25. The probe of claim 19, wherein the hybridization occurs under strict conditions.
